SEQUENCE LISTING (15 sequences)

SEQ ID NO: 1 - DNA, 40 bp, synthetic construct
  source 1..40: mol_type = other DNA
ggctcggccctgggtgaggggcgcagtggggtgtagccag

SEQ ID NO: 2 - DNA, 40 bp, synthetic construct
  source 1..40: mol_type = other DNA
gggcgtgtgtgggagggggcacttcgtcggggtgagccag

SEQ ID NO: 3 - DNA, 34 bp, synthetic construct
  source 1..34: mol_type = other DNA
ggctcggccctgggtgaggggcgcagtggggtgt

SEQ ID NO: 4 - DNA, 34 bp, synthetic construct
  source 1..34: mol_type = other DNA
gggcgtgtgtgggagggggcacttcgtcggggtg

SEQ ID NO: 5 - DNA, 34 bp, synthetic construct
  source 1..34: mol_type = other DNA
gggggccagtgaatgagttggggtggtaatgggg

SEQ ID NO: 6 - DNA, 34 bp, synthetic construct
  source 1..34: mol_type = other DNA
agagggggtggcgggtgtctggagtggagggtgg

SEQ ID NO: 7 - DNA, 34 bp, synthetic construct
  source 1..34: mol_type = other DNA
gggggttcacgctcgagggggccacagggggagt

SEQ ID NO: 8 - DNA, 47 bp, synthetic construct
  source 1..47: mol_type = other DNA
tgtaccgtctgagcgattcgtacnagccagtcagtgttaaggagtgc

SEQ ID NO: 9 - DNA, 23 bp, synthetic construct
  source 1..23: mol_type = other DNA
tgtaccgtctgagcgattcgtac

SEQ ID NO: 10 - DNA, 23 bp, synthetic construct
  source 1..23: mol_type = other DNA
gcactccttaacactgactggct

SEQ ID NO: 11 - DNA, 23 bp, synthetic construct
  source 1..23: mol_type = other DNA
gcactccttaacactgactggct

SEQ ID NO: 12 - DNA, 65 bp, synthetic construct
  source 1..65: mol_type = other DNA
  misc_feature 24..32: note = The feature in this nucleotide sequence denotes a xGen UDI-UMI adapter index i7
gatcggaagagcacacgtctgaactccagtcacnnnnnnnnatctcgtatgccgtcttctgcttg

SEQ ID NO: 13 - DNA, 70 bp, synthetic construct
  source 1..70: mol_type = other DNA
  misc_feature 24..32: note = The feature in this nucleotide sequence denotes a xGen UDI-UMI adapter index i5
aatgatacggcgaccaccgagatctacacnnnnnnnnacactctttccctacacgacgctcttccgatct

SEQ ID NO: 14 - DNA, 38 bp, synthetic construct
  source 1..38: mol_type = other DNA
agtcgttgggggggggggtgagcgttgcgcgcgtagcc

SEQ ID NO: 15 - DNA, 34 bp, synthetic construct; identical to SEQ ID NO: 4
  source 1..34: mol_type = other DNA
  misc_feature <1: note = The sequence contains a 5' Thiol Modifier C6 S-S conjugated to the 5' end of the DNA sequence.
  misc_feature >34: note = The sequence contains a 3AmMO a MMO probe anode conjugated to the 3' end of the DNA sequence.

(19) United States
(12) Patent Application Publication
(10) Pub. No.: US 2026/0071989 A1
(43) Pub. Date: Mar. 12, 2026

(54) SINGLE-STRANDED DNA MOLECULAR RECOGNITION ELEMENTS AGAINST CYANOTOXIN L-BMAA

(71) Applicant: University of Puerto Rico, San Juan, PR (US)

(72) Inventors: Eduardo Nicolau, San Juan, PR (US); Xaimara Santiago Maldonado, San Juan, PR (US); Jose Arcadio Rodriguez-Martinez, San Juan, PR (US)

(21) Appl. No.: 19/259,575

(22) Filed: Jul. 3, 2025

Related U.S. Application Data
(60) Provisional application No. 63/667,735, filed on Jul. 4, 2024.

(51) Int. Cl. G01N 27/327; C12N 15/115; G01N 33/543

(52) U.S. Cl. CPC G01N 27/3276; C12N 15/115; G01N 27/3277; G01N 27/3278; G01N 33/5438; C12N 2310/16; C12N 2310/531

(57) ABSTRACT

The present disclosure provides aptamers comprising a nucleotide sequence for binding β-N-methylamino-L-alanine (L-BMAA) or an isomer thereof as well as electrochemical aptamer-based sensor (EAB) compositions that include the aptamers. Further, methods of detecting β-N-methylamino-L-alanine (L-BMAA) or an isomer in a sample are also provided in order to identify presence of L-BMAA in the sample.

### CROSS-REFERENCE TO RELATED APPLICATIONS

This application claims the benefit under 35 U.S.C. § 119 (e) of U.S. Provisional Application Ser. No. 63/667,735, filed on Jul. 4, 2024, the entire disclosure of which is incorporated herein by reference.

### GOVERNMENT RIGHTS

This invention was made with government support under grant number 5R25GM061151-21 awarded by the Research Initiative by Scientific Enhancement (RISE), and the NASA MIRO-Puerto Rico Space Partnership for Research, Innovation, and Training (PR-SPRINT) under grant number 80NSSC19M0236. The government has certain rights in the invention.

### SEQUENCE LISTING

This application contains a Sequence Listing that has been submitted electronically in .xml format and is incorporated by reference in its entirety. Said .xml copy, created Jun. 30, 2025, is named “71900-427288 Sequence Listing” and is 16,498 bytes in size.

### BACKGROUND AND SUMMARY

Harmful algal bloom (HAB) is a phenomenon involving multiple species and classes of algae known to produce a variety of toxic and non-toxic species that can pose a threat to aquatic ecosystems and local economies as well as general public health. In recent decades, an increase in the frequency of HABs has been observed in coastal areas due to progressive warming of coastal waters and also non-climatic drivers such as increased nutrient runoff. In particular, HABs have been associated with multiple human poisoning syndromes related to the consumption of shellfish, fish, and other marine animals that accumulate toxic species in their bodies.

β-N-methylamino-L-alanine (L-BMAA) is noncanonical amino acid produced by cyanobacteria and eukaryotic microalgae in both water and terrestrial ecosystems. L-BMAA was discovered over 50 years ago on Guam, an island with a population exhibiting a 50-100 times higher prevalence of neurodegenerative diseases than the global average. For instance, Guamanian Amyotrophic Lateral Sclerosis/Parkinsonism Dementia Complex (ALS/PDCO) was later linked to the consumption of BMAA-contaminated foods such as cycad seed flour and flying foxes. As a result, L-BMAA has been the focus of numerous research studies that aim to understand its contribution in pathogenic processes of neurodegeneration. L-BMAA is also known to penetrate the blood-brain barrier and has been found on the brains of diseased patients who died from sporadic Amyotrophic Lateral Sclerosis and Alzheimer's disease. Moreover, the structural isomers of L-BMAA including N-(2-aminoethyl) glycine (AEG), β-amino-N-methylalanine (BAMA) and 2,4-diamonibutyric acid (DAB) often coexist in the same environments and are suggested to also exhibit neurotoxic effects in subjects.

Given the neurotoxic effects of L-BMAA and related isomers, the development of accurate methods to facilitate its detection in biological and environmental samples is imperative. However, current methods lack sufficient specificity for accurate identification of these harmful toxins. Therefore, there exists a need for new methodology to identify L-BMAA and its isomers in samples to prevent harmful effects in subject and in the environment.

Accordingly, the present disclosure provides compositions and related methods utilizing L-BMAA-specific DNA aptamers. The compositions and methods of the present disclosure provide several benefits compared to the current state of the art. In particular, the present disclosure presents innovative aptamer-based analytical techniques for L-BMAA analysis that impart significant impacts on environmental and food safety monitoring for the benefit of humans.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTIONS OF THE DRAWINGS

The detailed description particularly refers to the accompanying figures in which:

FIGS. 1A-1B show the chemical structures of the disclosure FIG. 1A depicts the chemical structure of BMAA FIG. 1B shows biotinylated BMAA including a biotin moiety with a PEG4 spacer arm covalently attached to the primary amine of BMAA.

FIGS. 2A-2D illustrate the chemical structures of counter targets, including isomers, as shown in FIG. 2A aminoethylglycine (AEG), 2,4-diaminobutyric acid (DAB) as shown in FIG. 2B, and other low molecular weight emerging contaminants Atenolol shown in FIG. 2C, and Mono-butyl phthalate shown in FIG. 2D.

FIG. 3 depicts a schematic representation of the in vitro selection using Bead-SELEX. During positive selection rounds, BMAA (dark spots) was incubated with the library pool. For the negative selection, the immobilization matrix with no BMAA (gray spheres) was incubated with the library pool. The counter selection involved the incubation of the library with structurally similar compounds (star, diamond).

FIG. 4 illustrates a schematic representation of the electrochemical aptamer-based (EAB) sensor fabrication. Aptamer BMAA_165_min was immobilized on gold (Au) electrodes through self-assembled monolayers (SAMs). Then, methylene blue (MB) was covalently linked to the aptamers via chemical cross-linking, and the electrode surface was blocked with 6-mercaptoetanol-1-hexanol 9 (MCH). Electrochemical detection of BMAA was measured using square wave voltammetry (SWV) and electrochemical impedance spectroscopy (EIS).

FIGS. 5A-5C show the results of sequencing analysis. FIG. 5A is a bar graph of the base distribution analysis of the relative frequency (%) of each nucleotide across different rounds of SELEX compared to the library control (R0). FIG. 5B shows a graph of the abundance (RPM) of the motifs predicted by AptaTRACE (GAGGGG, GGAGGG, GGGAG, GGGGG) and the random sequences chosen for comparison (AAAAA, CCCCC, CCTAGT, TCGATC). FIG. 5C is a bar graph of the enrichment values of the motifs defined as the abundance in R18 divided by the abundance in R0.

FIGS. 6A-6H depict the screening for L-BMAA binders using a SG fluorescence displacement assay. Binding isotherms of BMAA_159 (K_{d}=3.03) is shown in FIG. 6A, binding isotherm for BMAA_165 (K_{d}=0.234) shown in FIG. 6B, the binding isotherm for BMAA_172 (K_{d}=6.50) shown in FIG. 6C, the binding isotherm for BMAA_38 (K_{d}=18.6) in FIG. 6D. BMAA_96 did not show a concentration-dependent trend within the studied range of concentrations. The K_{d} values were determined through nonlinear regression analysis by fitting the data with one site-specific binding equation using GraphPad Prism 10.2.0. Control samples of BMAA_159, shown in FIG. 6E, BMAA_165 as in FIG. 6F, BMAA_172 shown in FIG. 6G, and BMAA_38 shown in FIG. 6H. Measurements were performed in triplicates and the error bars represent the calculated standard error.

FIGS. 7A-7D display the secondary structure of the aptamers with the highest affinity. The secondary structure of BMAA 159 is shown in FIG. 7A, the secondary structure of BMAA_165 shown in FIG. 7B, the secondary structure of BMAA_159_min shown in FIG. 7C and the secondary structure of BMAA_165_min is shown in FIG. 7D. Structures were predicted using the Mfold software at ambient temperature and ionic conditions of the selection buffer (100 mM NaCl and 2 mM MgCl₂).

FIGS. 8A-8F visualizes the affinity evaluation and characterization of aptamers. Evaluation of the binding affinity of full-length aptamers using the SG fluorescence displacement. Binding isotherms of BMAA_159 (K_{d}=2.2±0.1), BMAA_165 (K_{d}=0.32±0.02) and BMAA_165_scrambled (K_{d}=no binding) is shown in FIG. 8A. A Comparison of the fluorescence response of BMAA and structurally related compounds (AEG, DAB, atenolol) is shown in FIG. 8B. A full analysis of the independent experiments can be found in FIG. 11A and FIG. 11B, Table 6, of the binding affinity of truncated aptamers using BMAA-conjugated fluorescence assay. The binding isotherms of BMAA_159_min (K_{d}=6±1) and BMAA_165_min (K_{d}=0.63±0.02) are illustrated in FIG. 8C. A full analysis of the independent experiments can be found in FIG. 12A and FIG. 12B, Table 7. Measurements were performed in triplicate and the error bars represent the calculated standard error. The K_{d} values were determined through non-linear regression analysis by fitting the data with one site-specific binding equation using GraphPad Prism 10.2.0. Qualitative affinity evaluation of the truncated aptamers using the frequency intensity changes at the amino proton region measured with solution NMR. FIG. 8D shows the binding isotherms of BMAA_159_min and BMAA_165_min. The amino proton region of the ¹H NMR spectra is provided in FIG. 10A and FIG. 10B. Spectrums are the average of 2048 transients and error bars represent the signal/noise figure for each measurement. CD spectra of BMAA (10 μM), aptamers (5 μM), and aptamers incubated with BMAA (1:2 ratio) recorded in buffer (100 mM KCl, 5 mM MgCl₂ and 20 mM Tris, pH 7.6) the CD spectrum of BMAA_159_min shown in FIG. 8E and in FIG. 8F, the CD spectra of BMAA_165_min. Each CD spectra is an average of 5 scans.

FIG. 9 represents the non-specific binding of truncated aptamers measured with the BMAA conjugated fluorescence assay. Normalized fluorescence response of the aptamers (1 μM) with BMAA and without (control). Measurements were performed in triplicates and the error bars represent the calculated standard error.

FIGS. 10A-10B display the amino proton region of the ¹H NMR spectra recorded in 20 mM sodium phosphate buffer with 100 mM NaCl and 2 mM MgCl₂ at 700 MHz ¹H frequency of BMAA_159_min shown in FIG. 10A, and in FIG. 10B, BMAA_165_min. Aptamer solutions (20 μM) were incubated with increasing molar concentrations of BMAA. Chemical shift perturbations are highlighted, indicating a conformational change in the aptamer upon binding.

FIGS. 11A-11B illustrate independent experiments of the SG fluorescence displacement assay. Binding isotherms were obtained using GraphPad Prism 10.2.0 for BMAA_159 as shown in FIG. 11A and BMAA_165 shown in FIG. 11B. Measurements were performed in triplicates and the error bars represent the calculated standard error.

FIG. 12A-12B depict independent experiments of BMAA-conjugated fluorescence assay. Binding isotherms were obtained using GraphPad Prism 10.2.0 for BMAA_159 as in FIG. 12A, and in FIG. 12B BMAA_165. Measurements were performed in triplicates and the error bars represent the calculated standard error.

FIGS. 13A-13F show the electrochemical detection of BMAA. Characterization of Au electrodes before and after aptamer modification carried out in 5 mM [Fe(CN)₆]^{3-/4-} redox couple solution FIG. 13A show the CV and the Nyquist plot shown in FIG. 13B. Electrochemical detection of the EAB sensor upon addition of varying concentrations of BMAA (0, 1, 2, 5, 10, 20, 50, 100, 200, 500, 1000 pM) performed in buffer solution (4 mM NaCl, 0.2 mM MgCl₂, and 0.8 mM Tris at pH 7.4). FIG. 13C shows the changes in current measured with SWV. FIG. 13D depicts the analytical response using the absolute value of Z_{R} (Ω) obtained with EIS. FIG. 13E shows the calibration curve of the analytical response |Z_{R}| (Ω) against BMAA concentration (Y=90.77x+39, R=0.999, LOD=1.13±0.03, LOQ=1.46±0.07). Measurements represent the mean of three independent experiments (n=9) and the error bars represent the calculated standard error. A full analysis of the independent experiments can be found in the ESI (FIGS. 14A-14F and FIGS. 15A-15C, Table 8). FIG. 13F shows the comparison of the analytical response |Z_{R}| (Ω) of BMAA and structurally related compounds (AEG, DAB, atenolol). Measurements were performed in triplicates and the error bars represent the calculated standard error. (FIGS. 16A-16C).

FIGS. 14A-14F show the independent experiments for the electrochemical detection of the EAB sensor upon addition of varying concentrations of BMAA (0, 1, 2, 5, 10, 20, 50, 100, 200, 500, 1000 pM) performed in buffer solution (4 mM NaCl, 0.2 mM MgCl₂, and 0.8 mM Tris at pH 7.4). Changes in current measured using SWV electrode 1 is shown in FIG. 14A, electrode 2 in FIG. 14B, and electrode 3 in FIG. 14C. Analytical response using the absolute value of ZR (Ω) obtained using EIS. Electrode 1 is shown in FIG. 14D, Electrode 2 in FIG. 14E, and electrode 3 in FIG. 14F. Measurements were performed in triplicates.

FIGS. 15A-15C depict independent experiments of the EAB sensor. Calibration curves of the analytical response |ZR| (Ω) against BMAA concentration, shown in FIG. 15A for electrode 1 (Y=91x+45, R²=0.999, n=3), in FIG. 15B for electrode 2 (Y=92x+38, R²=0.999, n=3) and in FIG. 15C for electrode 3 (Y=89x+32, R²=0.999, n=3). Simple linear regression analysis was obtained using GraphPad Prism 10.2.0.

FIGS. 16A-16C show the electrochemical detection of the EAB sensor upon addition of varying concentrations of analyte (0, 1, 2, 5, 10, 20, 50, 100, 200, 500, 1000 pM) performed in buffer solution (4 mM NaCl, 0.2 mM MgCl₂, and 0.8 mM Tris at pH 7.4). Analytical response using the absolute value of ZR (52) obtained using EIS of the following analytes: AEG show in FIG. 16A, Atenolol, as in FIG. 16B, and DAB as in FIG. 16C. Measurements were performed in triplicate.

### DETAILED DESCRIPTION

Various embodiments of the invention are described herein as follows. In an illustrative aspect, an aptamer comprising a nucleotide sequence for binding β-N-methylamino-L-alanine (L-BMAA) or an isomer thereof is provided.

In an embodiment, the isomer is selected from the group consisting of (S)-2,4-diaminobutyric acid (L-DAB), N-(2-aminoethyl)glycine (AEG), and β-amino-N-methylalanine (BAMA). In an embodiment, the isomer is(S)-2,4-diaminobutyric acid (L-DAB). In an embodiment, the isomer is N-(2-aminoethyl)glycine (AEG). In an embodiment, the isomer is β-amino-N-methylalanine (BAMA).

In an embodiment, the nucleotide sequence is a DNA sequence. In an embodiment, the DNA sequence is single-stranded DNA (ssDNA). In an embodiment, the DNA sequence comprises a sequence motif of GAGGGG, GGAGGG, or both. In an embodiment, the DNA sequence has a tridentate secondary structure. In an embodiment, the DNA sequence comprises a thiol modification at its 5′ end. In an embodiment, the thiol modification is a 5′ Thio C6 modification.

In an embodiment, the DNA sequence comprises a probe at its 3′ end. In an embodiment, the probe comprises a 3′ amino modifier (3A). In an embodiment, the probe comprises a 3′ mixed metal oxide (mMO) coating. In an embodiment, the probe comprises a 3′ amino modifier (3A) and a 3′ mixed metal oxide (mMO).

In an embodiment, the DNA sequence comprises 80% sequence identity to SEQ ID NO: 1 (BMAA_159_min). In an embodiment, the DNA sequence comprises 85% sequence identity to SEQ ID NO: 1 (BMAA_159_min). In an embodiment, the DNA sequence comprises 90% sequence identity to SEQ ID NO: 1 (BMAA_159_min). In an embodiment, the DNA sequence comprises 91% sequence identity to SEQ ID NO: 1 (BMAA_159_min). In an embodiment, the DNA sequence comprises 92% sequence identity to SEQ ID NO: 1 (BMAA_159_min). In an embodiment, the DNA sequence comprises 93% sequence identity to SEQ ID NO: 1 (BMAA_159_min). In an embodiment, the DNA sequence comprises 94% sequence identity to SEQ ID NO: 1 (BMAA_159_min). In an embodiment, the DNA sequence comprises 95% sequence identity to SEQ ID NO: 1 (BMAA_159_min). In an embodiment, the DNA sequence comprises 96% sequence identity to SEQ ID NO: 1 (BMAA_159_min). In an embodiment, the DNA sequence comprises 97% sequence identity to SEQ ID NO: 1 (BMAA_159_min). In an embodiment, the DNA sequence comprises 98% sequence identity to SEQ ID NO: 1 (BMAA_159_min). In an embodiment, the DNA sequence comprises 99% sequence identity to SEQ ID NO: 1 (BMAA_159_min). In an embodiment, the DNA sequence comprises SEQ ID NO: 1 (BMAA_159_min). In an embodiment, the DNA sequence consists essentially of SEQ ID NO: 1 (BMAA_159_min). In an embodiment, the DNA sequence consists of SEQ ID NO: 1 (BMAA_159_min).

In an embodiment, SEQ ID NO: 1 (BMAA_159_min) has a B-DNA helix structure. In an embodiment, SEQ ID NO: 1 (BMAA_159_min) has a CD spectrum characterized by a negative peak at about 235 nm and a positive band at about 270-280 nm. In an embodiment, SEQ ID NO: 1 (BMAA_159_min) has a CD spectrum characterized by a positive band at about 270-280 nm. In an embodiment, SEQ ID NO: 1 (BMAA_159_min) has a CD spectrum characterized by a negative peak at about 235 nm.

In an embodiment, SEQ ID NO: 1 (BMAA_159_min) has a K_{d} between about 1 to about 10 μM for binding to L-BMAA. In an embodiment, the K_{d} is about 2 to about 9 μM. In an embodiment, the K_{d} is about 3 to about 8 μM. In an embodiment, the K_{d} is about 4 to about 7 μM. In an embodiment, the K_{d} is about 6±1 μM.

In an embodiment, the DNA sequence comprises 80% sequence identity to SEQ ID NO: 2 (BMAA_165_min). In an embodiment, the DNA sequence comprises 85% sequence identity to SEQ ID NO: 2 (BMAA_165_min). In an embodiment, the DNA sequence comprises 90% sequence identity to SEQ ID NO: 2 (BMAA_165_min). In an embodiment, the DNA sequence comprises 91% sequence identity to SEQ ID NO: 2 (BMAA_165_min). In an embodiment, the DNA sequence comprises 92% sequence identity to SEQ ID NO: 2 (BMAA_165_min). In an embodiment, the DNA sequence comprises 93% sequence identity to SEQ ID NO: 2 (BMAA_165_min). In an embodiment, the DNA sequence comprises 94% sequence identity to SEQ ID NO: 2 (BMAA_165_min). In an embodiment, the DNA sequence comprises 95% sequence identity to SEQ ID NO: 2 (BMAA_165_min). In an embodiment, the DNA sequence comprises 96% sequence identity to SEQ ID NO: 2 (BMAA_165_min). In an embodiment, the DNA sequence comprises 97% sequence identity to SEQ ID NO: 2 (BMAA_165_min). In an embodiment, the DNA sequence comprises 98% sequence identity to SEQ ID NO: 2 (BMAA_165_min). In an embodiment, the DNA sequence comprises 99% sequence identity to SEQ ID NO: 2 (BMAA_165_min). In an embodiment, the DNA sequence comprises SEQ ID NO: 2 (BMAA_165_min). In an embodiment, the DNA sequence consists essentially of SEQ ID NO: 2 (BMAA_165_min). In an embodiment, the DNA sequence consists of SEQ ID NO: 2 (BMAA_165_min).

In an embodiment, SEQ ID NO: 2 (BMAA_165_min) has a G-quadruplex structure. In an embodiment, the G-quadruplex structure is parallel. In an embodiment, the G-quadruplex structure is antiparallel. In an embodiment, the G-quadruplex structure is parallel and antiparallel. In an embodiment, SEQ ID NO: 2 (BMAA_165_min) has a CD spectrum characterized by a negative peak at about 235 nm and positive peaks at about 260 nm and about 290 nm. In an embodiment, SEQ ID NO: 2 (BMAA_165_min) has a CD spectrum characterized by a negative peak at about 235 nm. In an embodiment, SEQ ID NO: 2 (BMAA_165_min) has a CD spectrum characterized by a positive peak at about 260 nm. In an embodiment, SEQ ID NO: 2 (BMAA_165_min) has a CD spectrum characterized by a positive peak at about 290 nm. In an embodiment, SEQ ID NO: 2 (BMAA_165_min) has a CD spectrum characterized by positive peaks at about 260 nm and about 290 nm.

In an embodiment, SEQ ID NO: 2 (BMAA_165_min) has a K_{d} between about 0.1 to about 10 μM for binding to L-BMAA. In an embodiment, the K_{d} is about 0.1 to about 1 μM. In an embodiment, the K_{d} is about 0.2 to about 0.9 μM. In an embodiment, the K_{d} is about 0.4 to about 0.8 μM. In an embodiment, the K_{d} is about 0.5 to about 0.7 μM. In an embodiment, the K_{d} is about 0.6±0.02 μM.

In an embodiment, the DNA sequence has a dissociation constant (K_{d}) between about 0.1 to about 10 μM for binding to L-BMAA. In an embodiment, the K_{d} is about 0.1 to about 8 μM. In an embodiment, the K_{d} is less than 10 μM. In an embodiment, the K_{d} is less than 8 μM. In an embodiment, the K_{d} is less than 7 μM. In an embodiment, the K_{d} is less than 6 μM.

In an illustrative aspect, an electrochemical aptamer-based sensor (EAB) composition is provided. The EAB composition comprises a DNA aptamer and a gold nanoparticle, wherein the DNA aptamer is conjugated to the gold nanoparticle.

In an embodiment, the DNA aptamer is single-stranded DNA (ssDNA). In an embodiment, the DNA aptamer comprises a sequence motif of GAGGGG, GGAGGG, or both. In an embodiment, the DNA aptamer has a tridentate secondary structure. In an embodiment, the DNA sequence comprises a thiol modification at its 5′ end. In an embodiment, the thiol modification is a 5′ Thio C6 modification. In an embodiment, the DNA sequence comprises a probe at its 3′ end. In an embodiment, the probe comprises a 3′ amino modifier (3A). In an embodiment, the probe comprises a 3′ mixed metal oxide (mMO) coating. In an embodiment, the probe comprises a 3′ amino modifier (3A) and a 3′ mixed metal oxide (mMO).

In an embodiment, the DNA aptamer has a K_{d} for binding to L-BMAA of less than about 20 μM. In an embodiment, the DNA aptamer has a K_{d} for binding to L-BMAA of less than about 10 μM.

In an embodiment, the DNA aptamer is selected from the group consisting of SEQ ID NO: 1 (BMAA_159_min), SEQ ID NO: 2 (BMAA_165_min), SEQ ID NO: 3 (BMAA_159), SEQ ID NO: 4 (BMAA_165), SEQ ID NO: 5 (BMAA_172), SEQ ID NO: 6 (BMAA_96), and SEQ ID NO: 7 (BMAA_38). In an embodiment, the DNA aptamer comprises SEQ ID NO: 1 (BMAA_159_min).

In an embodiment, SEQ ID NO: 1 (BMAA_159_min) has a K_{d} between about 1 to about 10 UM for binding to L-BMAA. In an embodiment, the K_{d} is about 2 to about 9 μM. In an embodiment, the K_{d} is about 3 to about 8 μM. In an embodiment, the K_{d} is about 4 to about 7 μM. In an embodiment, the K_{d} is about 6±1 μM.

In an embodiment, the DNA aptamer comprises 80% sequence identity to SEQ ID NO: 1 (BMAA_159_min). In an embodiment, the DNA sequence comprises 85% sequence identity to SEQ ID NO: 1 (BMAA_159_min). In an embodiment, the DNA sequence comprises 90% sequence identity to SEQ ID NO: 1 (BMAA_159_min). In an embodiment, the DNA sequence comprises 91% sequence identity to SEQ ID NO: 1 (BMAA_159_min). In an embodiment, the DNA sequence comprises 92% sequence identity to SEQ ID NO: 1 (BMAA_159_min). In an embodiment, the DNA sequence comprises 93% sequence identity to SEQ ID NO: 1 (BMAA_159_min). In an embodiment, the DNA sequence comprises 94% sequence identity to SEQ ID NO: 1 (BMAA_159_min). In an embodiment, the DNA sequence comprises 95% sequence identity to SEQ ID NO: 1 (BMAA_159_min). In an embodiment, the DNA sequence comprises 96% sequence identity to SEQ ID NO: 1 (BMAA_159_min). In an embodiment, the DNA sequence comprises 97% sequence identity to SEQ ID NO: 1 (BMAA_159_min). In an embodiment, the DNA sequence comprises 98% sequence identity to SEQ ID NO: 1 (BMAA_159_min). In an embodiment, the DNA sequence comprises 99% sequence identity to SEQ ID NO: 1 (BMAA_159_min). In an embodiment, the DNA sequence comprises SEQ ID NO: 1 (BMAA_159_min). In an embodiment, the DNA sequence consists essentially of SEQ ID NO: 1 (BMAA_159_min). In an embodiment, the DNA sequence consists of SEQ ID NO: 1 (BMAA_159_min).

In an embodiment, the DNA aptamer comprises SEQ ID NO: 2 (BMAA_165_min). In an embodiment, SEQ ID NO: 2 (BMAA_165_min) has a K_{d} between about 0.1 to about 10 μM for L-BMAA. In an embodiment, the K_{d} is about 0.1 to about 1 μM. In an embodiment, the K_{d} is about 0.2 to about 0.9 μM. In an embodiment, the K_{d} is about 0.4 to about 0.8 μM. In an embodiment, the K_{d} is about 0.5 to about 0.7 μM. In an embodiment, the K_{d} is about 0.6±0.02 μM.

In an embodiment, the DNA aptamer comprises 80% sequence identity to SEQ ID NO: 2 (BMAA_165_min). In an embodiment, the DNA sequence comprises 85% sequence identity to SEQ ID NO: 2 (BMAA_165_min). In an embodiment, the DNA sequence comprises 90% sequence identity to SEQ ID NO: 2 (BMAA_165_min). In an embodiment, the DNA sequence comprises 91% sequence identity to SEQ ID NO: 2 (BMAA_165_min). In an embodiment, the DNA sequence comprises 92% sequence identity to SEQ ID NO: 2 (BMAA_165_min). In an embodiment, the DNA sequence comprises 93% sequence identity to SEQ ID NO: 2 (BMAA_165_min). In an embodiment, the DNA sequence comprises 94% sequence identity to SEQ ID NO: 2 (BMAA_165_min). In an embodiment, the DNA sequence comprises 95% sequence identity to SEQ ID NO: 2 (BMAA_165_min). In an embodiment, the DNA sequence comprises 96% sequence identity to SEQ ID NO: 2 (BMAA_165_min). In an embodiment, the DNA sequence comprises 97% sequence identity to SEQ ID NO: 2 (BMAA_165_min). In an embodiment, the DNA sequence comprises 98% sequence identity to SEQ ID NO: 2 (BMAA_165_min). In an embodiment, the DNA sequence comprises 99% sequence identity to SEQ ID NO: 2 (BMAA_165_min). In an embodiment, the DNA sequence comprises SEQ ID NO: 2 (BMAA_165_min). In an embodiment, the DNA sequence consists essentially of SEQ ID NO: 2 (BMAA_165_min). In an embodiment, the DNA sequence consists of SEQ ID NO: 2 (BMAA_165_min).

In an embodiment, the DNA aptamer comprises SEQ ID NO: 3 (BMAA_159).

In an embodiment, the DNA aptamer comprises SEQ ID NO: 4 (BMAA_165).

In an embodiment, the DNA aptamer comprises SEQ ID NO: 5 (BMAA_172). In an embodiment, SEQ ID NO: 5 (BMAA_172) has a K_{d} for binding to L-BMAA of about 6.5 μM.

In an embodiment, the DNA aptamer comprises SEQ ID NO: 6 (BMAA_96).

In an embodiment, the DNA aptamer comprises SEQ ID NO: 7 (BMAA_38). In an embodiment, SEQ ID NO: 5 (BMAA_172) has a K_{d} for binding to L-BMAA of about 18 μM.

In an embodiment, the EAB composition further comprises a redox label covalently linked to the DNA aptamer. In an embodiment, the redox label is methylene blue (MB).

In an embodiment, the DNA aptamer conjugated to the gold nanoparticle has an increase in resistance compared to the gold nanoparticle. In an embodiment, the DNA aptamer conjugated to the gold nanoparticle has a slower electron transfer rate compared to the gold nanoparticle. In an embodiment, the redox label reduces electron flow to the electrode on target binding.

In an illustrative aspect, a method of detecting β-N-methylamino-L-alanine (L-BMAA) or an isomer thereof in a sample is provided. The method comprises the steps of: a) contacting the sample with an aptamer comprising a nucleotide sequence for binding L-BMAA to provide a test sample, and b) performing an assay on the test sample to identify presence of L-BMAA in the sample.

In an embodiment, the isomer is selected from the group consisting of (S)-2,4-diaminobutyric acid (L-DAB), N-(2-aminoethyl)glycine (AEG), and β-amino-N-methylalanine (BAMA). In an embodiment, the isomer is(S)-2,4-diaminobutyric acid (L-DAB). In an embodiment, the isomer is N-(2-aminoethyl)glycine (AEG). In an embodiment, the isomer is β-amino-N-methylalanine (BAMA).

In an embodiment, the nucleotide sequence is a DNA sequence. In an embodiment, the DNA sequence is single-stranded DNA (ssDNA). In an embodiment, the DNA sequence comprises a sequence motif of GAGGGG (SEQ ID NO: 8), GGAGGG (SEQ ID NO: 9), or both. In an embodiment, the DNA sequence has a tridentate secondary structure.

In an embodiment, the DNA sequence comprises a thiol modification at its 5′ end. In an embodiment, the thiol modification is comprises a 5′ Thio C6 modification.

In an embodiment, the DNA sequence comprises a probe at its 3′ end. In an embodiment, the probe comprises a 3′ amino modifier (3A). In an embodiment, the probe comprises a 3′ mixed metal oxide (mMO) coating. In an embodiment, the probe comprises a 3′ amino modifier (3A) and a 3′ mixed metal oxide (mMO). In an embodiment, the DNA sequence is covalently linked to a redox label. In an embodiment, the redox label is methylene blue (MB).

In an embodiment, the DNA sequence comprises 80% sequence identity to SEQ ID NO: 1 (BMAA_159_min). In an embodiment, the DNA sequence comprises 85% sequence identity to SEQ ID NO: 1 (BMAA_159_min). In an embodiment, the DNA sequence comprises 90% sequence identity to SEQ ID NO: 1 (BMAA_159_min). In an embodiment, the DNA sequence comprises 91% sequence identity to SEQ ID NO: 1 (BMAA_159_min). In an embodiment, the DNA sequence comprises 92% sequence identity to SEQ ID NO: 1 (BMAA_159_min). In an embodiment, the DNA sequence comprises 93% sequence identity to SEQ ID NO: 1 (BMAA_159_min). In an embodiment, the DNA sequence comprises 94% sequence identity to SEQ ID NO: 1 (BMAA_159_min). In an embodiment, the DNA sequence comprises 95% sequence identity to SEQ ID NO: 1 (BMAA_159_min). In an embodiment, the DNA sequence comprises 96% sequence identity to SEQ ID NO: 1 (BMAA_159_min). In an embodiment, the DNA sequence comprises 97% sequence identity to SEQ ID NO: 1 (BMAA_159_min). In an embodiment, the DNA sequence comprises 98% sequence identity to SEQ ID NO: 1 (BMAA_159_min). In an embodiment, the DNA sequence comprises 99% sequence identity to SEQ ID NO: 1 (BMAA_159_min). In an embodiment, the DNA sequence comprises SEQ ID NO: 1 (BMAA_159_min). In an embodiment, the DNA sequence consists essentially of SEQ ID NO: 1 (BMAA_159_min). In an embodiment, the DNA sequence consists of SEQ ID NO: 1 (BMAA_159_min).

In an embodiment, SEQ ID NO: 1 (BMAA_159_min) has a B-DNA helix structure. In an embodiment, SEQ ID NO: 1 (BMAA_159_min) has a CD spectrum characterized by a negative peak at about 235 nm and a positive band at about 270-280 nm. In an embodiment, SEQ ID NO: 1 (BMAA_159_min) has a CD spectrum.

In an embodiment, SEQ ID NO: 1 (BMAA_159_min) has a K_{d} between about 1 to about 10 μM for binding to L-BMAA. In an embodiment, the K_{d} is about 2 to about 9 μM. In an embodiment, the K_{d} is about 3 to about 8 μM. In an embodiment, the K_{d} is about 4 to about 7 μM. In an embodiment, the K_{d} is about 6±1 μM.

In an embodiment, the DNA sequence comprises 80% sequence identity to SEQ ID NO: 2 (BMAA_165_min). In an embodiment, the DNA sequence comprises 85% sequence identity to SEQ ID NO: 2 (BMAA_165_min). In an embodiment, the DNA sequence comprises 90% sequence identity to SEQ ID NO: 2 (BMAA_165_min). In an embodiment, the DNA sequence comprises 91% sequence identity to SEQ ID NO: 2 (BMAA_165_min). In an embodiment, the DNA sequence comprises 92% sequence identity to SEQ ID NO: 2 (BMAA_165_min). In an embodiment, the DNA sequence comprises 93% sequence identity to SEQ ID NO: 2 (BMAA_165_min). In an embodiment, the DNA sequence comprises 94% sequence identity to SEQ ID NO: 2 (BMAA_165_min). In an embodiment, the DNA sequence comprises 95% sequence identity to SEQ ID NO: 2 (BMAA_165_min). In an embodiment, the DNA sequence comprises 96% sequence identity to SEQ ID NO: 2 (BMAA_165_min). In an embodiment, the DNA sequence comprises 97% sequence identity to SEQ ID NO: 2 (BMAA_165_min). In an embodiment, the DNA sequence comprises 98% sequence identity to SEQ ID NO: 2 (BMAA_165_min). In an embodiment, the DNA sequence comprises 99% sequence identity to SEQ ID NO: 2 (BMAA_165_min). In an embodiment, the DNA sequence comprises SEQ ID NO: 2 (BMAA_165_min). In an embodiment, the DNA sequence consists essentially of SEQ ID NO: 2 (BMAA_165_min). In an embodiment, the DNA sequence consists of SEQ ID NO: 2 (BMAA_165_min).

In an embodiment, SEQ ID NO: 2 (BMAA_165_min) has a G-quadruplex structure. In an embodiment, the G-quadruplex structure is parallel. In an embodiment, the G-quadruplex structure is antiparallel. In an embodiment, the G-quadruplex structure is parallel and antiparallel. In an embodiment, SEQ ID NO: 2 (BMAA_165_min) has a CD spectrum characterized by a negative peak at about 235 nm and positive peaks at about 260 nm and about 290 nm. In an embodiment, SEQ ID NO: 2 (BMAA_165_min) has a CD spectrum characterized by a negative peak at about 235 nm. In an embodiment, SEQ ID NO: 2 (BMAA_165_min) has a CD spectrum characterized by a positive peak at about 260 nm. In an embodiment, SEQ ID NO: 2 (BMAA_165_min) has a CD spectrum characterized by a positive peak at about 290 nm. In an embodiment, SEQ ID NO: 2 (BMAA_165_min) has a CD spectrum characterized by positive peaks at about 260 nm and about 290 nm.

In an embodiment, SEQ ID NO: 2 (BMAA_165_min) has a K_{d} between about 0.1 to about 10 UM for binding to L-BMAA. In an embodiment, the K_{d} is about 0.1 to about 1 μM. In an embodiment, the K_{d} is about 0.2 to about 0.9 μM. In an embodiment, the K_{d} is about 0.4 to about 0.8 μM. In an embodiment, the K_{d} is about 0.5 to about 0.7 μM. In an embodiment, the K_{d} is about 0.6±0.02 μM.

In an embodiment, the DNA sequence has a dissociation constant (K_{d}) between about 0.1 to about 10 UM for binding to L-BMAA. In an embodiment, the K_{d} is about 0.1 to about 8 μM. In an embodiment, the K_{d} is less than 10 μM. In an embodiment, the K_{d} is less than 8 μM. In an embodiment, the K_{d} is less than 7 μM. In an embodiment, the K_{d} is less than 6 μM.

In an embodiment, the sample is selected from the group consisting of an environmental sample, a tissue sample, a food sample, a cyanobacterial sample, a microalgae sample, a plant sample, a zooplankton sample, an animal sample, and a supplement sample.

In an embodiment, the environmental sample is selected from the group consisting of a freshwater sample, a marine water sample, a brackish water sample, and a terrestrial sample.

In an embodiment, the sample is a cyanobacterial sample, wherein the cyanobacterial is selected from the group consisting of Anabaena, Anabaenopsis, Aphanizomenon, Calothrix, Chlorogloeopsis, Chroococcidiopsis, Cyanobium, Cylindrospermopsis, Fischerella, Gomphosphaeria, Leptolyngbya, Lyngbya, Microcoleus, Microcystis, Myxosarcina, Nodularia, Nostoc, Oscillatoria, Phormidium, Planktothrix, Plectonema, Prochlorococcus, Pseudanabaena, Scytonema, Symploca, Synechococcus, Synechocystis, Trichodesmium, and Woronichinia.

In an embodiment, the sample is an animal sample, wherein the animal sample is selected from the group consisting of a mollusk, a crustacean, a fish, a bird, and a mammals.

In an embodiment, the sample is a plant sample, wherein the plant sample is selected from the group consisting of Azolla filiculoides, Brassica oleracea, Cycas micronesica, Cycas revoluta, Cycas debaoensis, Gunnera kauaiensi, and Lathyrus latifolius.

In an embodiment, the assay comprises square wave voltammetry (SWV), electrochemical impedance spectroscopy (EIS), or both. In an embodiment, the assay is SWV and comprises movement of a redox label away from an electrode upon L-BMAA binding. In an embodiment, the assay is SWV and comprises a decrease in electron flow to an electrode upon L-BMAA binding. In an embodiment, the assay is EIS and comprises a change in impedance upon L-BMAA binding.

In an illustrative aspect, a second method of detecting β-N-methylamino-L-alanine (L-BMAA) or an isomer thereof in a sample is provided. The method comprises the steps of: a) contacting the sample with an electrochemical aptamer-based sensor (EAB) composition comprising a DNA aptamer and a gold nanoparticle, wherein the DNA aptamer is conjugated to the gold nanoparticle to provide a test sample, and b) performing an assay on the test sample to identify presence of L-BMAA in the sample.

In an embodiment, the isomer is selected from the group consisting of (S)-2,4-diaminobutyric acid (L-DAB), N-(2-aminoethyl)glycine (AEG), and β-amino-N-methylalanine (BAMA). In an embodiment, the isomer is(S)-2,4-diaminobutyric acid (L-DAB). In an embodiment, the isomer is N-(2-aminoethyl)glycine (AEG). In an embodiment, the isomer is β-amino-N-methylalanine (BAMA).

In an embodiment, the DNA aptamer is single-stranded DNA (ssDNA). In an embodiment, the DNA sequence comprises a sequence motif of GAGGGG, GGAGGG, or both. In an embodiment, the DNA aptamer has a tridentate secondary structure.

In an embodiment, the DNA sequence comprises a thiol modification at its 5′ end. In an embodiment, the thiol modification comprises a 5′ Thio C6 modification. In an embodiment, the DNA sequence comprises a probe at its 3′ end. In an embodiment, the probe comprises a 3′ amino modifier (3A). In an embodiment, the probe comprises a 3′ mixed metal oxide (mMO) coating. In an embodiment, the probe comprises a 3′ amino modifier (3A) and a 3′ mixed metal oxide (mMO).

In an embodiment, the DNA aptamer has a K_{d} for L-BMAA of less than about 20 μM. In an embodiment, the DNA aptamer has a K_{d} for L-BMAA of less than about 10 μM.

In an embodiment, the DNA aptamer is selected from the group consisting of SEQ ID NO: 1 (BMAA_159_min), SEQ ID NO: 2 (BMAA_165_min), SEQ ID NO: 3 (BMAA_159), SEQ ID NO: 4 (BMAA_165), SEQ ID NO: 5 (BMAA_172), SEQ ID NO: 6 (BMAA_96), and SEQ ID NO: 7 (BMAA_38).

In an embodiment, the DNA aptamer comprises SEQ ID NO: 1 (BMAA_159_min). In an embodiment, the DNA aptamer comprises 80% sequence identity to SEQ ID NO: 1 (BMAA_159_min) or SEQ ID NO: 2 (BMAA_165_min). In an embodiment, the DNA aptamer comprises 80% sequence identity to SEQ ID NO: 1 (BMAA_159_min). In an embodiment, the DNA sequence comprises 85% sequence identity to SEQ ID NO: 1 (BMAA_159_min). In an embodiment, the DNA sequence comprises 90% sequence identity to SEQ ID NO: 1 (BMAA_159_min). In an embodiment, the DNA sequence comprises 91% sequence identity to SEQ ID NO: 1 (BMAA_159_min). In an embodiment, the DNA sequence comprises 92% sequence identity to SEQ ID NO: 1 (BMAA_159_min). In an embodiment, the DNA sequence comprises 93% sequence identity to SEQ ID NO: 1 (BMAA_159_min). In an embodiment, the DNA sequence comprises 94% sequence identity to SEQ ID NO: 1 (BMAA_159_min). In an embodiment, the DNA sequence comprises 95% sequence identity to SEQ ID NO: 1 (BMAA_159_min). In an embodiment, the DNA sequence comprises 96% sequence identity to SEQ ID NO: 1 (BMAA_159_min). In an embodiment, the DNA sequence comprises 97% sequence identity to SEQ ID NO: 1 (BMAA_159_min). In an embodiment, the DNA sequence comprises 98% sequence identity to SEQ ID NO: 1 (BMAA_159_min). In an embodiment, the DNA sequence comprises 99% sequence identity to SEQ ID NO: 1 (BMAA_159_min). In an embodiment, the DNA sequence comprises SEQ ID NO: 1 (BMAA_159_min). In an embodiment, the DNA sequence consists essentially of SEQ ID NO: 1 (BMAA_159_min). In an embodiment, the DNA sequence consists of SEQ ID NO: 1 (BMAA_159_min).

In an embodiment, SEQ ID NO: 1 (BMAA_159_min) has a K_{d} between about 1 to about 10 μM for binding to L-BMAA. In an embodiment, the K_{d} is about 2 to about 9 μM. In an embodiment, the K_{d} is about 3 to about 8 μM. In an embodiment, the K_{d} is about 4 to about 7 μM. In an embodiment, the K_{d} is about 6±1 μM.

In an embodiment, the DNA aptamer comprises SEQ ID NO: 2 (BMAA_165_min). In an embodiment, the DNA aptamer comprises 80% sequence identity to SEQ ID NO: 2 (BMAA_165_min). In an embodiment, the DNA sequence comprises 85% sequence identity to SEQ ID NO: 2 (BMAA_165_min). In an embodiment, the DNA sequence comprises 90% sequence identity to SEQ ID NO: 2 (BMAA_165_min). In an embodiment, the DNA sequence comprises 91% sequence identity to SEQ ID NO: 2 (BMAA_165_min). In an embodiment, the DNA sequence comprises 92% sequence identity to SEQ ID NO: 2 (BMAA_165_min). In an embodiment, the DNA sequence comprises 93% sequence identity to SEQ ID NO: 2 (BMAA_165_min). In an embodiment, the DNA sequence comprises 94% sequence identity to SEQ ID NO: 2 (BMAA_165_min). In an embodiment, the DNA sequence comprises 95% sequence identity to SEQ ID NO: 2 (BMAA_165_min). In an embodiment, the DNA sequence comprises 96% sequence identity to SEQ ID NO: 2 (BMAA_165_min). In an embodiment, the DNA sequence comprises 97% sequence identity to SEQ ID NO: 2 (BMAA_165_min). In an embodiment, the DNA sequence comprises 98% sequence identity to SEQ ID NO: 2 (BMAA_165_min). In an embodiment, the DNA sequence comprises 99% sequence identity to SEQ ID NO: 2 (BMAA_165_min). In an embodiment, the DNA sequence comprises SEQ ID NO: 2 (BMAA_165_min). In an embodiment, the DNA sequence consists essentially of SEQ ID NO: 2 (BMAA_165_min). In an embodiment, the DNA sequence consists of SEQ ID NO: 2 (BMAA_165_min).

In an embodiment, SEQ ID NO: 2 (BMAA_165_min) has a K_{d} between about 0.1 to about 10 μM for binding to L-BMAA. In an embodiment, the K_{d} is about 0.1 to about 1 μM. In an embodiment, the K_{d} is about 0.2 to about 0.9 μM. In an embodiment, the K_{d} is about 0.4 to about 0.8 μM. In an embodiment, the K_{d} is about 0.5 to about 0.7 μM. In an embodiment, the K_{d} is about 0.6±0.02 μM.

In an embodiment, the DNA aptamer comprises SEQ ID NO: 3 (BMAA_159).

In an embodiment, the DNA aptamer comprises SEQ ID NO: 4 (BMAA_165).

In an embodiment, the DNA aptamer comprises SEQ ID NO: 5 (BMAA_172). In an embodiment, SEQ ID NO: 5 (BMAA_172) has a K_{d} for L-BMAA of about 6.5 μM.

In an embodiment, the DNA aptamer comprises SEQ ID NO: 6 (BMAA_96).

In an embodiment, the DNA aptamer comprises SEQ ID NO: 7 (BMAA_38). In an embodiment, SEQ ID NO: 5 (BMAA_172) has a K_{d} for L-BMAA of about 18 μM.

In an embodiment, the sample is selected from the group consisting of an environmental sample, a tissue sample, a food sample, a cyanobacterial sample, a microalgae sample, a plant sample, a zooplankton sample, an animal sample, and a supplement sample.

In an embodiment, the environmental sample is selected from the group consisting of a freshwater sample, a marine water sample, a brackish water sample, and a terrestrial sample.

In an embodiment, the sample is a cyanobacterial sample, wherein the cyanobacterial is selected from the group consisting of Anabaena, Anabaenopsis, Aphanizomenon, Calothrix, Chlorogloeopsis, Chroococcidiopsis, Cyanobium, Cylindrospermopsis, Fischerella, Gomphosphaeria, Leptolyngbya, Lyngbya, Microcoleus, Microcystis, Myxosarcina, Nodularia, Nostoc, Oscillatoria, Phormidium, Planktothrix, Plectonema, Prochlorococcus, Pseudanabaena, Scytonema, Symploca, Synechococcus, Synechocystis, Trichodesmium, and Woronichinia.

In an embodiment, the sample is an animal sample, wherein the animal sample is selected from the group consisting of a mollusk, a crustacean, a fish, a bird, and a mammals.

In an embodiment, the sample is a plant sample, wherein the plant sample is selected from the group consisting of Azolla filiculoides, Brassica oleracea, Cycas micronesica, Cycas revoluta, Cycas debaoensis, Gunnera kauaiensi, and Lathyrus latifolius.

In an embodiment, the assay comprises square wave voltammetry (SWV), electrochemical impedance spectroscopy (EIS), or both. In an embodiment, the assay is SWV and comprises movement of a redox label away from an electrode upon L-BMAA binding. In an embodiment, the assay is SWV and comprises a decrease in electron flow to an electrode upon L-BMAA binding. In an embodiment, the assay is EIS and comprises a change in impedance upon L-BMAA binding.

In an embodiment, the DNA aptamer conjugated to the gold nanoparticle has an increase in resistance compared to the gold nanoparticle. In an embodiment, the DNA aptamer conjugated to the gold nanoparticle has a slower electron transfer rate compared to the gold nanoparticle. In an embodiment, the redox label reduces electron flow to the electrode on target binding.

### EXAMPLES

### Example 1

### Materials and Methods

### Materials

All DNA oligos and DNA library Prep MC UNI Kit with full-length indexed adapters (xGen UDI-UMI adapters) were obtained from Integrated DNA Technologies (Coralville, IA, USA) (Table 1 and Table 2). Biotinylated BMAA was synthesized by Pepscan (Lelystad, Netherlands). Dynabeads™ M-280 Streptavidin was acquired from Invitrogen (Carlsbad, CA, USA). Econotag PLUS 2× Master Mix (pH 9.0, 0.1 units per μL of EconoTaq DNA polymerase, 400 UM dNTPs, 3 mM MgCl₂) was from Lucigen (Middleton, WI, USA). Polymerase chain reaction (PCR) purification was achieved using a QIAquick PCR Purification Kit from Qiagen (Germantown, MD, USA). DNA purification from agarose gels was performed with the QIAquick Gel Extraction Kit from Qiagen (Germantown, MD, USA). Lambda Exonuclease was purchased from New England Biolabs (NEB; Ipswich, MA).

Sodium chloride, magnesium chloride, 1 M Tris-HCl (pH=8), streptavidin-coated agarose resin, glycogen, molecular-grade ethanol, mono-butyl phthalate, atenolol, S(+)-2-amino-3-(methylamino) propionic acid hydrochloride (1-BMAA, ≥97% NMR), aminoethylglycine (AEG), 2,4-aminobutyric acid (DAB), SYBR® Green I nucleic acid gel stain (10 000× in DMSO), Multiscreen® 96-well plates (flat bottom clear and black), Nuclease-Free Water (Molecular Biology Grade), Tris(2-carboxyethyl) phosphine hydrochloride (TCEP), potassium hexacyanoferrate (iii), potassium hexacyanoferrate (ii), Methylene Blue (Atto MB2 NHS ester), and 6-mercapto-1-hexanol were purchased directly from Sigma Aldrich (St. Louis, MO, USA). Invitrogen™ SYBR™ Gold Nucleic Acid Gel Stain (10 000× concentrate in DMSO), Pierce™ Streptavidin Coated Immunoassay Plates (96-well black) were purchased from Thermo Fisher Scientific (Waltham, MA, USA). Flow-through columns were from Bio-Rad Laboratories, Inc. (Hercules, CA, USA). Gold (Au) electrode 3.0 mm diameter, coiled platinum wire auxiliary electrode (23 cm), and RE-5B Ag/AgCl (3 M NaCl) reference electrode were purchased from Bioanalytical Systems, Inc. (West Lafayette, IN, USA). Nanopore water (18.2 MΩ cm², Milli-Q Direct 16) was used unless otherwise stated.

TABLE 1
DNA oligos used for SELEX

| Name | Sequence (5′-3′) |
|---|---|
| Library | TGTACCGTCTGAGCGATTCGTAC(N₃₄) |
| | AGCCAGTCAGTGTTAAGGAGTGC |
| | (SEQ ID NO: 8) |
| Forward primer | TGTACCGTCTGAGCGATTCGTAC |
| | (SEQ ID NO: 9) |
| Forward primer + | /Biotin/GCACTCCTTAACACTGAC |
| biotin | TGGCT (SEQ ID NO: 10) |
| Reverse primer | GCACTCCTTAACACTGACTGGCT |
| | (SEQ ID NO: 11) |
| xGen full-length | GATCGGAAGAGCACACGTCTGAAC |
| UDI-UMI adapter | TCCAGTCAC [i7]₈ UMI |
| index i7 | ATCTCGTATGCCGTCTTCTGCTTG |
| | (SEQ ID NO: 12) |
| xGen full-length | AATGATACGGCGACCACCGAGAT |
| UDI-UMI adapter | CTACAC [i5]₈ |
| index i5 | ACACTCTTTCCCTACACGACGCT |
| | CTTCCGATCT (SEQ ID NO: 13) |

TABLE 2
Sequences of the DNA aptamers (overrepresented
motifs are underlined)

| Full length DNA | |
|---|---|
| aptamers | N₃₄ Sequence (5′-3′) |
| BMAA_159 | GGCTCGGCCCTGGGTGAGGGGCGC |
| | AGTGGGGTGT (SEQ ID NO: 3) |
| BMAA_165 | GGGCGTGTGTGGGAGGGGGCACTT |
| | CGTCGGGGTG (SEQ ID NO: 4) |
| BMAA_172 | GGGGGCCAGTGAATGAGTTGGGGT |
| | GGTAATGGGG (SEQ ID NO: 5) |
| BMAA_96 | AGAGGGGGTGGCGGGTGTCTGGAG |
| | TGGAGGGTGG (SEQ ID NO: 6) |
| BMAA_38 | GGGGGTTCACGCTCGAGGGGGCCAC |
| | AGGGGGAGT (SEQ ID NO: 7) |
| BMAA_165_ | AGTCGTTGGGGGGGGGGGTGAGCGT |
| scrambled | TGCGCGCGTAGCC (SEQ ID |
| | NO: 14) |
| Truncated DNA | |
| aptamers | Sequence (5′-3′) |
| BMAA_159_min | GGCTCGGCCCTGGGTGAGGGGCG |
| | CAGTGGGGTGTAGCCAG (SEQ |
| | ID NO: 1) |
| BMAA_165_min | GGGCGTGTGTGGGAGGGGGCACT |
| | TCGTCGGGGTGAGCCAG (SEQ |
| | ID NO: 2) |
| Biosensing DNA | |
| aptamer | Sequence (5′-3′) |
| BMAA_165_min | /5ThioMC6-D/GGGCGTGTGTGG |
| | GAGGGGGCACTTCGTCGGGGTG/ |
| | 3AmMO/ (SEQ ID NO: 15) |

### In Vitro Selection of Aptamers

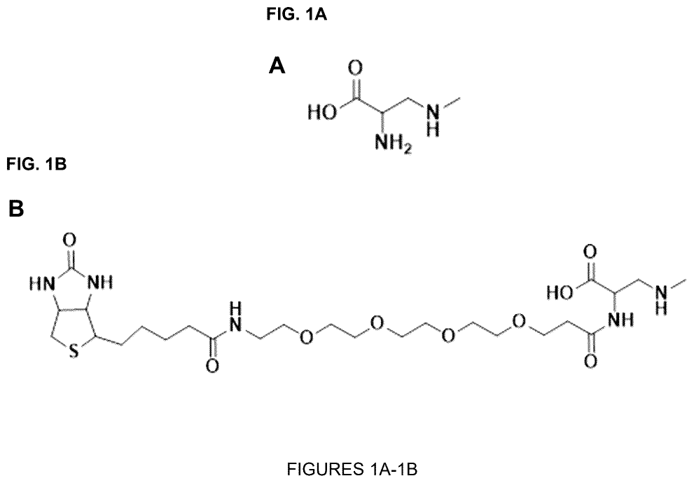

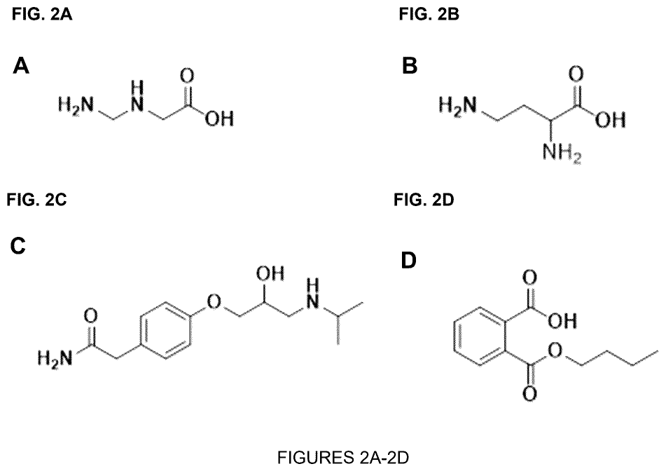

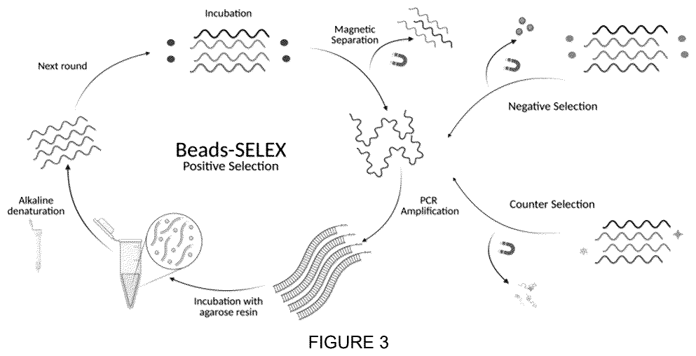

A selection buffer (SB) composed of 100 mM NaCl, 5 mM MgCl₂, and 20 mM Tris (pH 7.6) was used throughout the selection. Before each selection round, aptamers were heated to 90° C. for 5 minutes and cooled at room temperature for 15 minutes. All binding reactions had a total volume of 500 μL and were incubated with rotation at room temperature. Biotinylated BMAA (FIG. 1B) was dissolved in SB to a concentration of 50 μM and immobilized onto streptavidin magnetic beads, according to the manufacturer's directions, to form the immobilization target (IT). For the first round of positive selection (R1), the IT was incubated with 2000 pmoles of ssDNA library at equal molar ratios for 48 hours. Following incubation, unbound ssDNAs were removed by magnetic separation, washed 3 times with SB, and resuspended in 200 μL of SB. Subsequent selection rounds were performed using 200 pmoles of ssDNA pool and incubated with equal molar ratios of the target. The same process was followed for positive selections through round 5 with decreasing incubation times. For the first round of negative selection (R4), the biotinylated linker (no target) was immobilized onto streptavidin magnetic beads, forming the immobilization substrate (IS). This was incubated with the ssDNA pool for 1 hour. Following incubation, magnetic separation was used to recover the supernatant with the unbound sequences. Supernatants from 3 consecutive washes with SB were also recovered to serve as a template for amplification. The same process was followed for the remaining negative selections with increasing incubation times. Competitive elution (CE) rounds (R6-R11) included an incubation of IT followed by magnetic separation and resuspension in a free BMAA (FIG. 1A) solution. As in the negative selections, the supernatant was collected to serve as a template for amplification. The target concentration and incubation times were decreased each round. Finally, negative selection rounds from R10 to R16 included an incubation with IT followed by incubation with structurally similar targets (FIGS. 2A-2D). Then, the beads were washed and resuspended in SB to be used as templates for amplification. Detailed selection conditions are provided in Table 3 and a schematic representation of the process detailed in FIG. 3.

TABLE 3
Beads-SELEX scheme for selection of BMAA-specific aptamers

| | Positive | | | Negative | |
|---|---|---|---|---|---|
| Round | Selection | Time | Round | Selection | Time |
| 1 | IT | 48 hr. | | — | — |
| 2 | IT | 24 hr. | | — | — |
| 3 | IT | 18 hr. | 4 | IS | 1 hr. |
| 5 | IT | 12 hr. | 6 | IS | 3 hr. |
| 7 | IT | 6 hr. | 8 | IS | 6 hr. |
| 9 | IT/CE | 3 hr./ | 10 | IT/1 μM | 3 hr./ |
| | 500 μM | 1 hr. | | CTs | 1.5 hr./ |
| | BMAA | | | | 1.5 hr. |
| 11 | IT/CE | 1 hr. 30 min. | 12 | IT/1 μM | 3 hr./ |
| | 500 μM | | | IM AEG | 3 hr. |
| | BMAA | | | | |
| 13 | IT/CE | 30 min./ | 14 | IT/1 μM | 3 hr./ |
| | 100 μM | 15 min. | | IM L-DAB | 3 hr. |
| | BMAA | | | | |
| 15 | IT/CE | 15 min./ | 16 | IT/1 μM | 1 hr./ |
| | 10 μM | 5 min. | | Ims | 1 hr./ |
| | BMAA | | | (serial) | 1 hr. |
| 17 | IT/CE | 5 min./ | | — | — |
| | 1 μM | 2 min. | | | |
| | BMAA | | | | |
| 18 | IT/CE | 1 min./ | | — | — |
| | 100 nM | 1 min. | | | |
| | BMAA | | | | |

(IT = immobilization target, IS = immobilization substrate, CE = competitive elution, CTs = counter targets, IM = isomers).

PCR reactions were carried out using a T100 Thermal Cycler from Bio-Rad Laboratories, Inc. (Hercules, CA) with a primer concentration of 400 nM. Thermal cycling conditions were 95° C. for 5 minutes [95° C. for 1 minute, 6° C. for 45 seconds, 72° C. for 1 minute] and a final extension of 72° C. for 7 minutes. For every selection round, low-volume (50 μL) PCR reactions were carried out with varying numbers of cycles and analyzed in 4% agarose gels to determine the optimal number of PCR cycles. Then, large-scale PCR reactions (4 mL) were performed using the determined number of cycles and purified using the QIAquick PCR Purification Kit. The concentration of dsDNA was measured with NanoDrop One/One Microvolume UV-Vis Spectrophotometer from (Thermo Fisher, Waltham, MA, USA). Products were analyzed in 4% agarose gels stained with ethidium bromide (120 V for 40 minutes). Gel images were obtained using the Azure Sapphire Biomolecular Imager (Azure Biosystems, Inc., Dublin, CA, USA). When required, purifications from 2% agarose gels were achieved by cutting the band of interest with a razor and extracting dsDNA following the QIAquick Gel Extraction Kit protocol.

For the generation of ssDNA, streptavidin agarose resin was incubated with dsDNA (100 μL of resin per 50 μg of dsDNA) for 2 hr. at room temperature with rotation. After incubation, the mixture was transferred to a flow-through column and washed with 2.5 mL of PBS 1×. Then, ssDNA was eluted with 2 mL of a 20 mM NaOH solution in triplicate (using the same flow-through). The ssDNA was ethanol precipitated using 10 mg mL⁻¹ of glycogen, 0.1 volumes of ammonium acetate (3 M, pH 5.2), and 3 volumes of ethanol at −80° C. for 1 hr. Afterward, the samples were centrifuged at 4° C. for 60 min., dried, and resuspended in SB. The ssDNA concentration was determined using the NanoDrop and the product was analyzed on a denaturing urea PAGE (8% polyacrylamide 7 M urea, 1.0 mm, 90 V for 1.5 hours), followed by staining with SYBR Gold for at least 30 min. Gel images were obtained using the Azure Sapphire Biomolecular Imager. If necessary, gel purification was carried out following the standard crush and soak method. Briefly, the desired band was excised from the gel, crushed, and extracted overnight with a buffer containing 10 mM magnesium acetate tetrahydrate, 0.5 M ammonium acetate, 1 mM EDTA (pH 8.0) at 30° C., followed by ethanol precipitation as previously described.

### Sequencing and Analysis

Ten samples were sent for high-throughput sequencing (HTS). Two samples corresponded to the original library. From SELEX, rounds 3, 8, 9, 12, 13, 16, 17, and 18 were selected. The libraries corresponding to each round were amplified using PCR. The purified products were used as DNA templates for the library preparation method. End-repair and adapter ligation were carried out using full-length adapter sequences with unique dual indexes (UDIs). After ligation, the fully indexed products were purified using a QIAquick PCR Purification Kit, pooled into one sample, and purified from 2% agarose gel. The dsDNA concentration was measured with a Qubit 4 Fluorometer using a 1× dsDNA HS (high sensitivity) assay kit (Invitrogen, Carlsbad, CA, USA). An aliquot of 50 μL from the PCR sample was sent for high-throughput sequencing using the Illumina HiSeq PE150 system (Novogene Corporation Inc., Sacramento, CA, USA).

Cutadapt and Trimmomatic were used as trimming tools for HTS data pre-processing. The first analysis of HTS data was achieved using the AptaSUITE pipeline via the graphical user interface (GUI). Base distribution analysis across the selection and preliminary analysis of the sequence distributions and enrichment were achieved with this platform. Within AptaSUITE, the AptaTRACE algorithm identified possible sequence-structure binding motifs (k-mer size of 6) for both Beads-SELEX and GO-SELEX. The GUI of the FASTAptamerR 2.0 pipeline was used for sequence counts, sequence enrichment, clustering, motif analysis across populations, data filtration, and plotting. Enrichment is defined as the normalized values (reads per million) of the last round of selection (R18) divided by the normalized values of the library control (R0). Secondary structures, free energies (AGs), and melting temperatures (Tₘ) were predicted using the Mfold software.

### SYBR Green (SG) Fluorescence Displacement Assay

The SYBR Green (SG) assay was modified from previous studies. SG (2×) and target dilutions were prepared in SB. Aptamers were heated to 90° C. for 5 min., then 4 μL of aptamer (10 μM) were mixed with 4 μL SG 2× and incubated for 30 min. at room temperature with rotation. Then, 8 μL of the mixture was transferred to a black 96-well plate, with wells containing 117 μL varying concentrations of BMAA (0, 0.049, 0.098, 0.20, 0.39, 0.78, 1.6, 3.1, 6.3, 13, 25 μM), for a total volume of 125 μL. Control samples included SG 2×, target, and aptamer alone, as well as target-SG and target-aptamer mixtures. Fluorescence was measured in an Infinite 200 PRO microplate reader (Tecan, Männedorf, Switzerland) using an excitation wavelength of 480 nm and an emission wavelength of 520 nm. Fluorescence at 520 nm was used to calculate the signal response:

$$Response ( R ) = \frac{{F}_{θ} - F}{F} \qquad Equation 1$$

where F_{θ} is the fluorescence without a target and F is at a given concentration of the target. The dissociation constant (K_{d}) was determined through nonlinear regression analysis by fitting the data with one site-specific binding equation. Experiments were performed in triplicate. Cross-reactivity assays were made similarly but using a fixed analyte concentration of 12.5 μM.

### BMAA-Conjugated Fluorescence Assay

Streptavidin-coated 96-well plates were used to conjugate biotinylated BMAA. Aptamer and target dilutions were prepared in SB. Before the experiment, aptamers were heated to 90° C. for 5 minutes and cooled for 15 minutes at room temperature. First, each well was washed with 200 μL of SB 3 times. Then, 100 μL of 10 μM biotinylated BMAA was added to each well and incubated for 2 h at room temperature. The wells were washed with 200 μL of SB 3 times. Aliquots of 100 μL of FAM-labeled aptamer solutions at varying concentrations (0, 0.08, 0.16, 0.32, 0.63, 1.3, 2.5, 5.0, 10, 20 μM) were added to the wells and incubated for 30 minutes at room temperature with shaking. Finally, the wells were washed with 200 μL of SB 3 times and resuspended in 100 μL of SB. For the negative controls, 1 μM FAM-labeled aptamers were added to previously washed wells (no target) and incubated for 30 minutes at room temperature. Fluorescence was measured in an Infinite 200 PRO microplate reader with an excitation wavelength of 480 nm and emission at 520 nm. Fluorescence response was calculated using equation 2:

$$Response ( R ) = \frac{{F}_{θ} - F}{F} \qquad Equation 2$$

The dissociation constant (K_{d}) was determined through nonlinear regression analysis by fitting the data with one site-specific binding equation. All samples were prepared in triplicates.

### Nuclear Magnetic Resonance

Solution NMR spectra were recorded on a Bruker AVANCE III HD spectrometer operating at a ¹H Larmor frequency of 500 mHz a Prodigy BBO Cryoprobe (Bruker, Billerica, MA, USA). One-dimensional excitation sculpting ¹H spectra were acquired at 298 K with a 2 second recycle delay, 45.3 us dwell time, and a 1.49 ms acquisition period. All samples were dissolved in 20 mM sodium phosphate buffer with 100 mM NaCl and 2 mM MgCl₂. All NMR samples included 10% D₂O. Before NMR analysis, aptamers were heated to 90° C. for 5 min. and cooled for 15 minutes at room temperature. For the titration experiments, a series of spectra were recorded upon the addition of aliquots containing molar equivalents in steps of 0.0625:1, up to 3:1 ligand:aptamer, according to the concentration of each aptamer (20 μM BMAA_159 and 14.4 μM BMAA_165). Each spectrum was the average of 2048 transients. Changes in frequency intensity at the amino proton region (7.93 ppm for BMAA_159 and 7.83 ppm for BMAA_165) were plotted against the molar fraction of the target to create binding isotherms.

### Circular Dichroism Spectroscopy

Samples including 10 μM BMAA, 10 μM aptamers (full-length and minimers), and aptamer/target mixtures (1:1 molar ratio, 30 minutes incubation at room temperature) were prepared in SB. Circular dichroism (CD) spectra were recorded on a Jasco J-810 CD spectropolarimeter (Jasco, Inc., Easton, MD) from 320 to 210 nm in a 1 mm path-length quartz cuvette at room temperature. Data gathered were the average of 5 scans collected in units of millidegrees (mdeg) versus wavelength at a scanning rate of 100 nm min-1. Scan of SB buffer alone was subtracted from the average scans of all samples.

### Electrochemical Detection of BMAA

All electrochemical measurements were done using a three-electrode electrochemical cell with an Au electrode (bare or aptamer-modified) as the working electrode, a platinum wire as the counter electrode, and an Ag/AgCl reference electrode. A Reference 600+ Gamry potentiostat was used for the electrochemical measurements, and the Gamry Instruments Framework software was used for data collection. The cyclic voltammetry (CV) experiments were performed by scanning between-0.2 and 0.6 V at a scan rate of 50 mV s⁻¹. For EIS, an amplitude of 10 mV, an initial frequency of 5 MHZ, and a final frequency of 10 Hz were used. SWV was measured in a potential window between −0.4 and 0 V with a pulse size of 25 mV and a frequency of 25 Hz. Before modification, Au electrodes were polished in silica slurry of various particle sizes (1.0, 0.3, and 0.05 μm) and washed with nanopure water. The electrodes were electrochemically cleaned in 0.1 M H₂SO₄ using CV by scanning between 0 and 1.6 V for 250 cycles, then rinsed with nanopure water. For aptamer immobilization, 2 μL of 100 μM aptamer and 40 μL of 5 mM TCEP were incubated for 1 h. Clean Au electrodes were incubated with 10 μL of the aptamer/TCEP mixture overnight, then rinsed with nanopure water. Electrochemical characterization of electrodes before and after aptamer modification was carried out in 5 mM [Fe(CN)_{6]}^{3-/4-} redox couple solution (1:1 molar ratio) in 0.1 M KCl using CV and EIS.

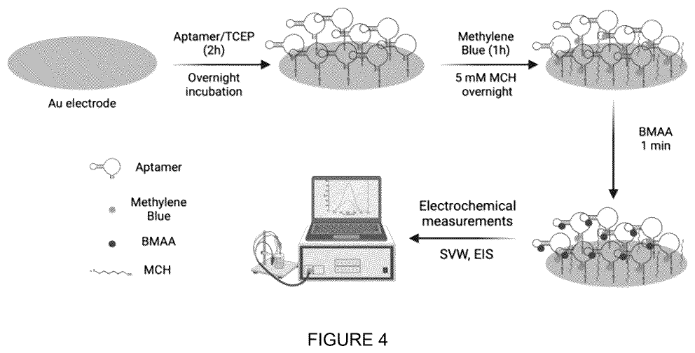

The aptamer-modified Au electrodes were incubated with 10 μL 1 mM MB solution for 1 h to link MB to aptamers covalently. Afterward, electrodes were rinsed with nanopure water and incubated with 10 μL of 5 mM 6-mercaptoetanol-1-hexanol 9 (MCH) solution overnight. A buffer composed of 4 mM NaCl, 0.2 mM MgCl₂, and 0.8 mM Tris at pH 7.4 was used for all biosensing experiments and solutions. A 1 μM BMAA master solution was freshly prepared before each experiment and used to prepare standard solutions of 100, 10, and 1 nM for titration into the electrochemical cell. The buffer volume in the electrochemical cell was 15 mL. The detection of BMAA was attained by adding increasing aliquots of target to achieve concentrations between 1 and 1000 μM. Then, the electrochemical cell was stirred for 1 minute without disturbing the electrodes. This was followed by a waiting period of 30 s to be certain the solution was stagnant. Electrochemical measurements were performed using SWV and EIS. Cross-reactivity experiments were carried out following the same procedure but using AEG, DAB, or atenolol as analytes. The resistive impedance (Z_{R}) was used to investigate the biosensor's analytical response using EIS. For this, the EIS spectra of the blank (no analyte) were subtracted from all samples using Gamry Echem Analyst 2 software. Calibration curves were fitted using standard linear regression. The limit of detection (L.O.D) and limit of quantification (L.O.Q) were calculated using equation 3 and equation 4, respectively, where S is the slope of the calibration curve and σ is the standard deviation (SD). FIG. 4 provides a schematic representation of EAB sensor fabrication and the electrochemical detection of BMAA.

$$L . O . D . = \frac{3.3 σ}{S} \qquad Equation 3$$

$$L . O . Q . = \frac{10 σ}{S} \qquad Equation 4$$

### Example 2

### In Vitro Selection

The Beads-SELEX approach was developed for the application of binding L-BMAA. Magnetic beads were used as a solid support to facilitate the separation of target-bound sequences. Negative selection, counter selection, and competitive elution rounds were included at various stages of selection to account for immobilization-related limiting factors like steric hindrance and nonspecific binding. The library input and incubation times were gradually decreased during positive selection rounds to increase the stringency of selection while minimizing the loss of low-frequency high-affinity oligonucleotides. Negative selection rounds were introduced to minimize non-specific binders to the immobilization matrix. The incubation times for the negative selection were continually increased during SELEX to intensify the rigorousness of selection. Competitive elution rounds were added to isolate sequences with a higher affinity towards the free target than the immobilized target. To further promote the enrichment of highly specific aptamers, counter selection rounds were used to select against structurally similar compounds (FIGS. 2A-2D). These counter targets included small molecules atenolol and mono-butyl phthalate, both of which fall under the category of contaminants of emerging concern. Likewise, as BMAA and its isomers frequently coexist in heterogeneous environments, AEG and DAB were added in subsequent counter selection rounds.

### Sequencing Analysis

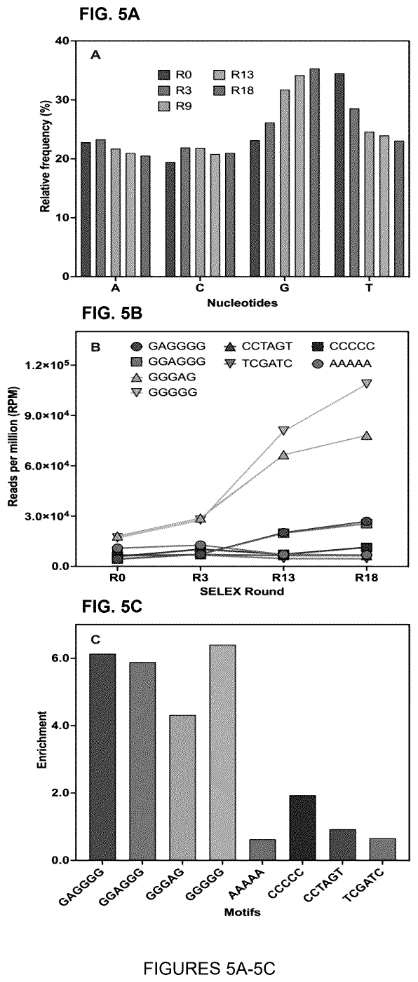

The identification of aptamer candidates is essential to the success of any SELEX process. Thousands or millions of sequences in each library can be elucidated through high-throughput sequencing (HTS), making it more suitable for large SELEX library pools than the standard Sanger sequencing. Many factors can result in high-abundance sequences that might not possess the highest binding capabilities, including PCR bias, non-uniform sequence distribution, energy-driven selection, and non-specific binding. Since the best binders may be underrepresented in the pool, selecting sequences based on their maximal abundance is not always ideal. Using a variety of bioinformatic techniques, HTS analysis was carried out at various stages of selection to study changes in the library pools. First, the pool composition from several selection cycles were examined and compared to the original library. The base composition of the initial library was 23% A, 19% C, 23% G, and 35% T (FIG. 5A). During SELEX, a 2% frequency decrease in the A content was observed while the C content increased by 2%. The G content showed a 12% frequency increase from the start to the end of the selection along with a 12% decrease in the T content (FIG. 5A). These shifts in base composition suggest the evolution of G-rich sequences during SELEX.

Subsequently, the library pools were examined to identify binding motifs. These are short sequences typically responsible for target binding, and they are often the ones conserved and enriched throughout selection. Once identified, binding motifs can help reduce the pool diversity for further stages of analysis. Four overrepresented 6-mer motifs were identified: TGGGGG, GGGGAG, GAGGGG, and GGAGGG (Table 4).

TABLE 4
Identification of overrepresented
motifs using AptaTRACE

| Selec- | | | Seed | Seed | Motif |
|---|---|---|---|---|---|
| tion | Motif | | P- | frequency | frequency |
| method | profile | Seed | value | (%) | (%) |
| Beads- | GGGGG | TGGGGG | 0.050 | 3.93 | 8.29 |
| SELEX | GGGAG | GGGGAG | 0.045 | 2.87 | 4.44 |
| | GAGGGG | GAGGGG | 0.031 | 2.69 | 2.69 |
| | GGAGGG | GGAGGG | 0.029 | 2.54 | 2.54 |

To validate these findings, the motifs' frequencies were monitored during several stages of the selection process and their total enrichment was determined. Every motif displayed an increase in abundance that peaked between R3 and R13 and continued to rise until the end of selection (FIG. 5B). This was likely due to the increasing stringency of conditions with the addition of negative and counter selection rounds. A variety of random motifs were included for comparison to ensure that the findings are a direct outcome of the selection process. The enrichment values of all identified motifs were higher than those of the random motifs (FIG. 5C). These results support the findings obtained from the population analysis and establish a connection between the enrichment of sequences with conserved G-rich motifs and the notable changes in base distribution throughout SELEX. High G-content is a common feature in several DNA aptamers, including other small-molecule binding DNA aptamers, and is often associated with the potential to adopt G-quadruplex (G4) structures. The enrichment of these motifs might indicate that G-rich sequences are preferred for BMAA binding.

To reduce the pool diversity and narrow down possible candidates, the sequences were clustered. Representative sequences were chosen from each cluster and their secondary structures were predicted. A total of five aptamer candidates were chosen to screen for binders based on consensus families. The sequences of these candidates are summarized in Table 2.

### Example 3

### Affinity Evaluation and Characterization of Aptamers

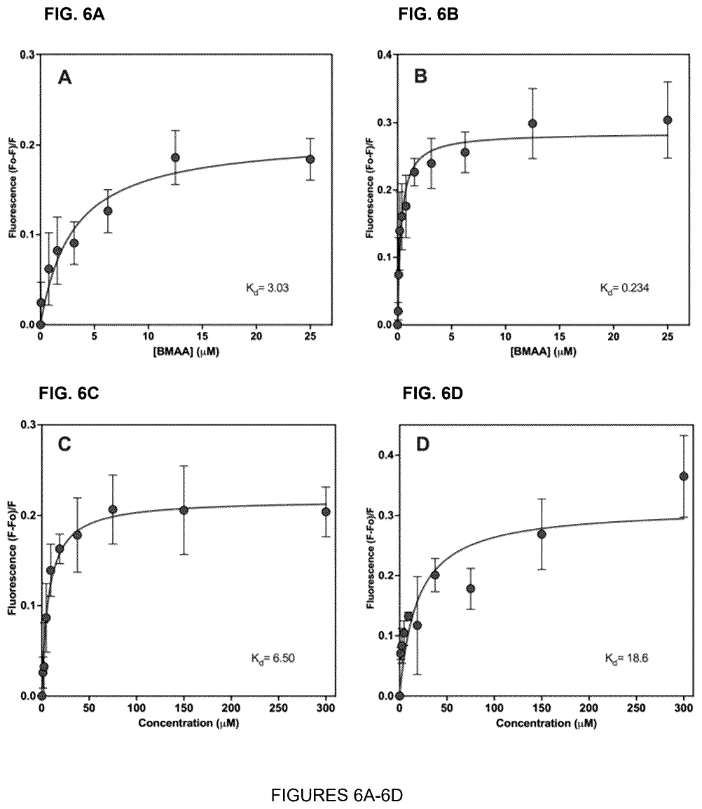

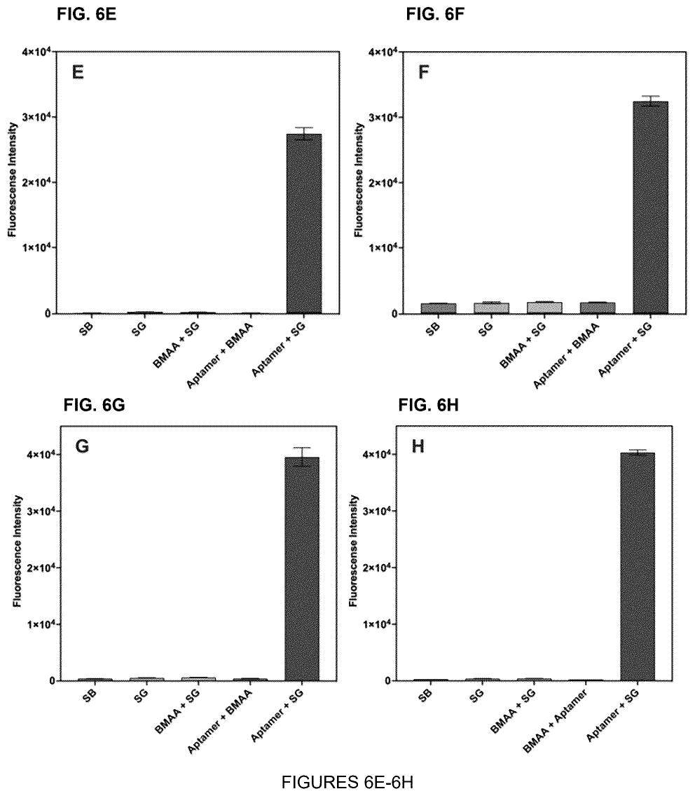

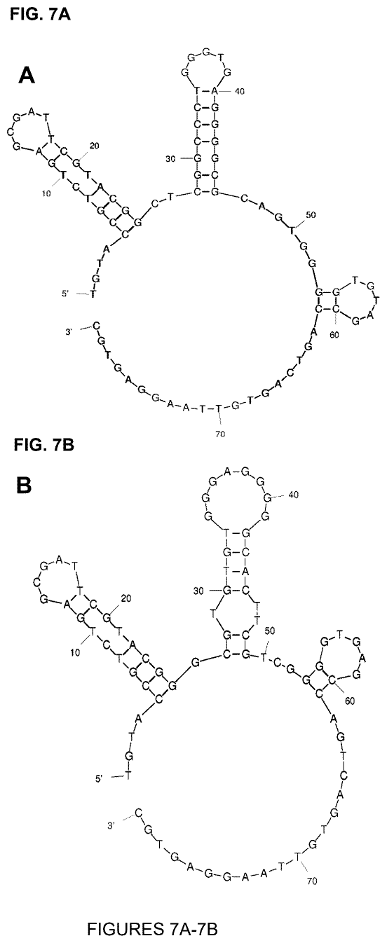

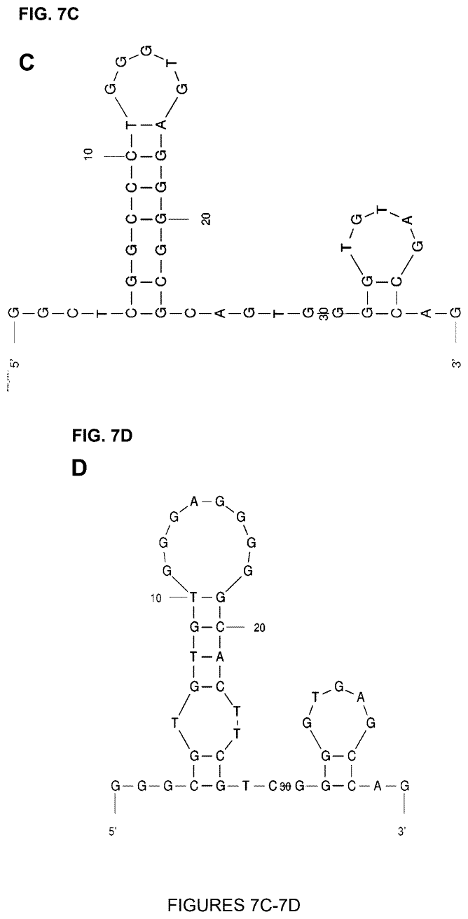

Given there is no universal characterization method suitable for all aptamers, various techniques were employed to confirm aptamer-target binding. The SG fluorescence displacement assay enables accurate determination of the dissociation constant (K_{d}) and rapid screening of binders. When SG intercalates in the hybridized regions of the aptamers, fluorescence is substantially increased, and aptamer-target interactions cause detectable changes in fluorescence intensity. This assay was used for screening the selected full-length aptamer candidates. Control experiments showed that fluorescence enhancement was only observed in the presence of aptamers (FIGS. 6A-6H). Changes in fluorescence were measured following the addition of various BMAA concentrations. Four candidates displayed BMAA binding, with affinities under 20 μM (FIGS. 6A-6H). Candidates BMAA_159 and BMAA_165 were selected for additional characterization due to their lower K_{d} values. In both cases, fluorescence decreased as target concentration increased, suggesting that SG was displaced as a result of target binding (FIG. 6A). Fitting of the binding curves using one site-specific binding showed affinities of 2.2±0.1 μM for BMAA_159 and 0.32±0.02 UM for BMAA_165. A higher binding affinity was observed for BMAA_165. Notably, the aptamers BMAA_159 and BMAA_165 contained the motifs GAGGGG and GGAGGG, respectively, and have similar secondary structures (FIGS. 7A-7D). The random region of BMAA_165 was scrambled to provide a control ssDNA sequence (Table 2).

The lack of response supports the involvement of these motifs and the resulting secondary structures in BMAA binding (FIG. 6A). The motifs may interact directly with BMAA or stabilize the bases directly engaged in BMAA binding. Additionally, the SG assay was used to study the cross-reactivity of the aptamers against potentially interfering compounds. When exposed to BMAA, both aptamers exhibited a considerably higher fluorescence response than the other compounds tested (FIG. 6B). This shows a high degree of selectivity and highlights the significance of negative selection rounds involving possibly interfering substances during the selection process. In summary, aptamer characterization with the SG assay demonstrated that both BMAA_159 and BMAA_165 exhibit high target specificity and selectivity.

Aptamers BMAA_159 and BMAA_165 were truncated into 40-base long structures (Table 2) with secondary structures characterized by two stem loops (FIGS. 7A-7D). Advantages of truncated aptamers include higher production yield, lower cost, and occasionally, lower K_{d} values.

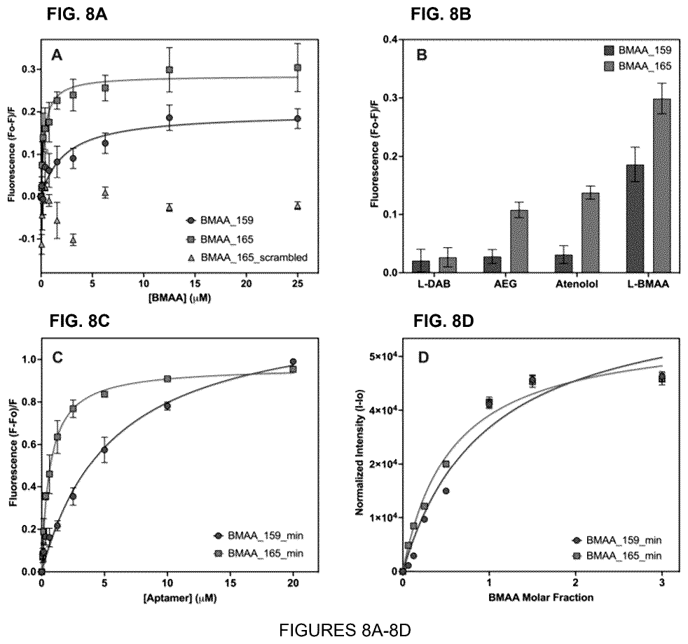

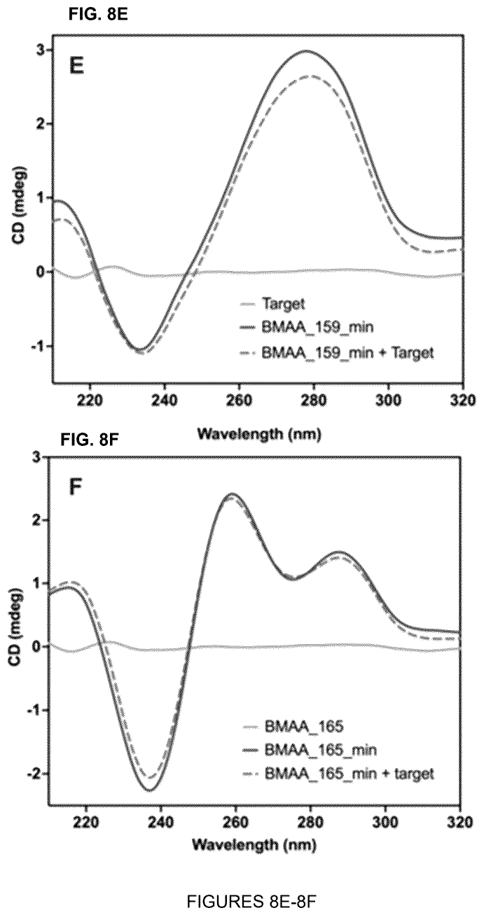

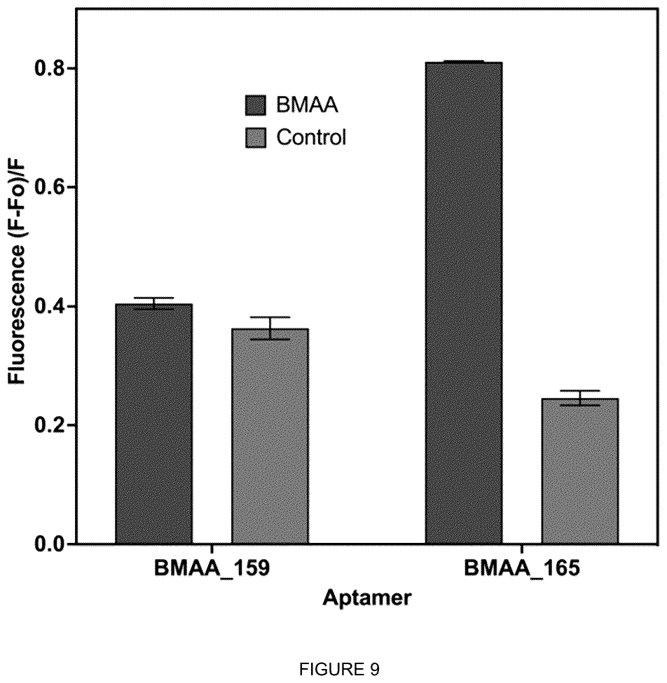

An alternative characterization method was explored to evaluate binding affinity. For this assay, BMAA was conjugated on streptavidin-coated 96-well plates and exposed to varying concentrations of FAM-labeled aptamers. It resembles the standard affinity chromatography technique as it involves target immobilization on a solid support. An increase in fluorescence intensity was observed with increasing aptamer concentrations (FIG. 8C). The apparent K_{d} values for BMAA_159_min and BMAA 165_min were 6±1 μM and 0.63±0.02 μM, respectively. Variations in affinity measurements could be observed when employing different characterization techniques due to intrinsic differences among methods. Particularly, the higher K_{d} values obtained with this assay can be a result of a decreased affinity of the aptamers towards the immobilized target. It is widely recognized that non-specific binding can occur in affinity measurements where the target is immobilized. The fluorescence response with and without BMAA was examined to assess non-specific binding. When BMAA was present, the fluorescence response was higher for both aptamers (FIG. 9). The difference in fluorescence response was higher for BMAA_165_min when compared to BMAA_159_min. Accordingly. BMAA_165_min exhibits a lower degree of non-specific adsorption in addition to a higher binding affinity.

TABLE 5
Apparent K_{d} values for the DNA aptamers.

| | K_{d} | K_{d} |
|---|---|---|
| ID | (95% CI) | (μM) |
| BMAA_159 | 1.02-8.66 | 3.03 |
| BMAA_165 | 0.149-0.690 | 0.234 |
| BMAA_172 | 5.23-9.56 | 6.50 |
| BMAA_96 | No binding* | — |
| BMAA_38 | 3.08-102 | 18.6 |

Apparent K_{d} values of the initial screening were obtained using the SG fluorescence displacement assay. The K_{d} values were determined through non-linear regression analysis by fitting the data with one site-specific binding equation using GraphPad Prism 10.2.0. No binding refers to the absence of a concentration-dependent trend within the studied range of concentrations.

TABLE 6
Apparent K_{d} values for the DNA aptamers.

| | | K_{d} | | K_{d} | |
|---|---|---|---|---|---|
| | K_{d} | (μM) | | (μM) | Standard |
| Aptamer | (95% CI) | best fit | R² | mean | error |
| | 0.93-4.38 | 2.06 | 0.94 | | |
| BMAA_159 | 0.78-6.50 | 2.30 | 0.90 | 2.2 | 0.1 |
| | 0.84-5.91 | 2.27 | 0.92 | | |
| | 0.60-1.24 | 0.86 | 0.98 | | |
| BMAA_165 | 0.18-0.37 | 0.26 | 0.98 | 0.32 | 0.02 |
| | 0.20-0.51 | 0.32 | 0.97 | | |

Apparent K_{d} values were obtained using the SG fluorescence displacement assay. Each measurement represents an independent experiment. The K_{d} values were determined through non-linear regression analysis by fitting the data with one site-specific binding equation using GraphPad Prism 10.2.0.

TABLE 7
Apparent K_{d} values for the minimized DNA aptamers.

| | | K_{d} | | K_{d} | |
|---|---|---|---|---|---|
| | K_{d} | (μM) | | (μM) | Standard |
| Aptamer | (95% CI) | best fit | R² | mean | error |
| BMAA_159_min | 3.12-8.26 | 5.02 | 0.98 | 6 | 1 |
| | 2.55-7.03 | 4.20 | 0.98 | | |
| | 4.53-17.72 | 8.54 | 0.97 | | |
| BMAA_165_min | 0.56-0.75 | 0.65 | 0.99 | 0.63 | 0.02 |
| | 0.52-0.66 | 0.58 | 0.99 | | |
| | 0.55-0.76 | 0.65 | 0.99 | | |

Apparent K_{d} values were obtained using the BMAA-conjugated fluorescence assay. Each measurement represents an independent experiment. The K_{d} values were determined through non-linear regression analysis by fitting the data with one site-specific binding equation using GraphPad Prism 10.2.0.

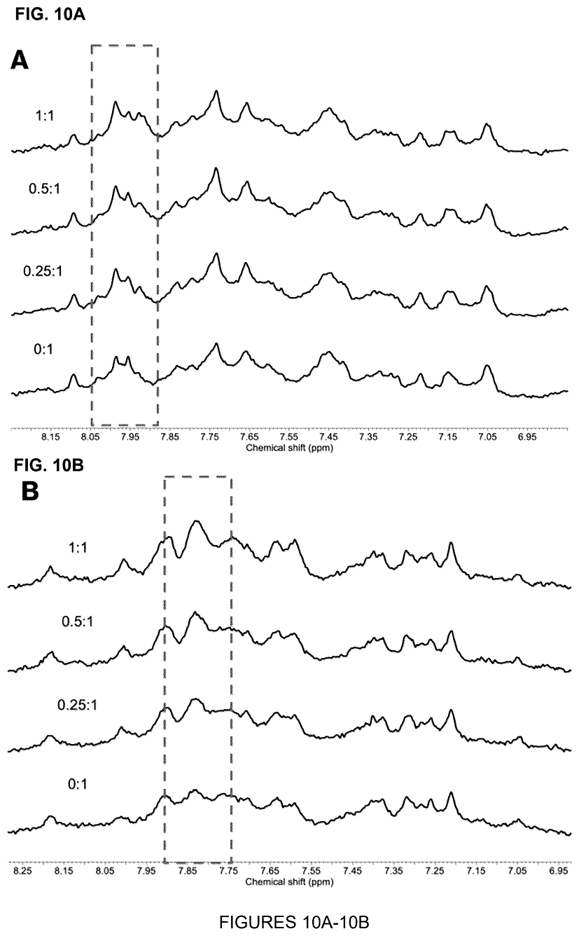

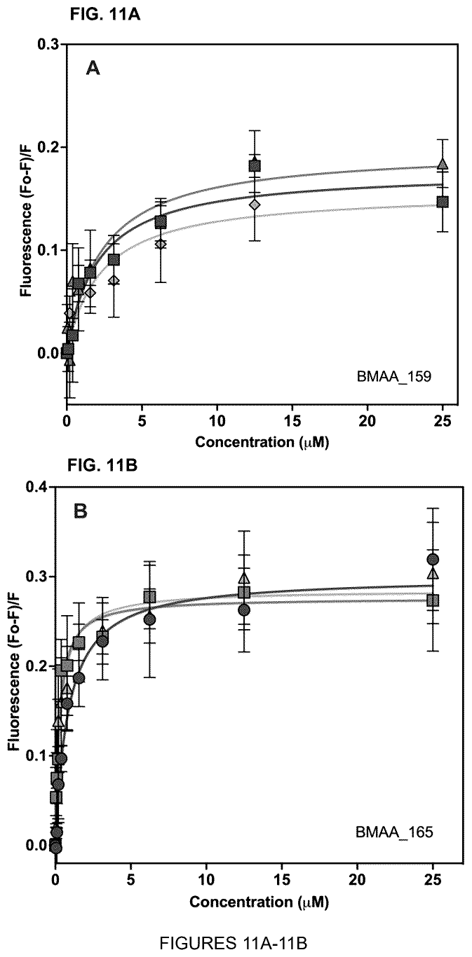

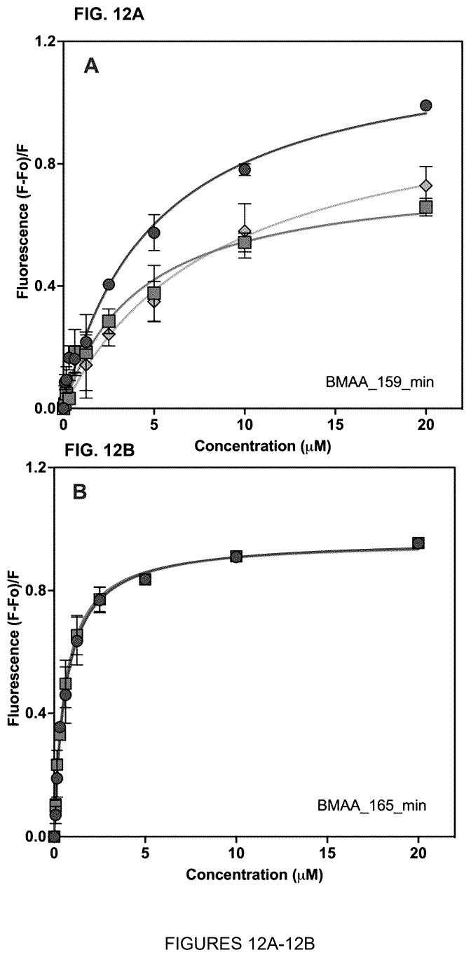

Solution NMR was used to further confirm the truncated aptamers' binding affinity along with obtaining structural and dynamic details about the aptamer-target systems. The amino proton region of the NMR spectra was used for the analysis since the ¹H NMR signals arising from the nitrogen bases are sensitive to conformational changes and hydrogen bonding interactions. Each aptamer showed characteristic chemical shift perturbations upon incubation with BMAA. For aptamer BMAA_159_min, the most significant chemical shift perturbations are observed at 7.12 ppm and 7.93 ppm (FIG. 10A). For BMAA_165_min, the most significant chemical shift perturbations are observed at 7.62 and 7.83 ppm (FIG. 10B). When plotted, the changes in chemical shift follow a concentration-dependent behavior (FIG. 8D). This phenomenon points to the formation of new hydrogen bonds or conformational changes during BMAA binding. Aptamer BMAA_165_min achieved saturation at lower BMAA concentrations, which correlates with a higher affinity towards the target. The results were consistent with the BMAA binding patterns seen in previous studies.

Further, CD spectroscopy was used to investigate the secondary structures of the truncated aptamers with and without BMAA. The CD spectrum of BMAA_159_min (FIG. 8E) showed a negative band at 240 nm and a positive band at 275 nm, characteristic of B-form DNA structures. A reduction in the CD amplitude was observed after incubation with BMAA. Prior research has linked this amplitude reduction to a transition from a B-form duplex to a hairpin. These stem-loop structures can be linked to target binding and additional stabilization to the secondary structure. The CD spectrum of BMAA_165_min showed a negative peak at 235 nm and positive peaks at 260 nm and 290 nm (FIG. 8F). These peaks were consistent with the formation of hybrid G4 structures (parallel and antiparallel). Following BMAA incubation, no significant changes in ellipticity were observed, suggesting that the hybrid G4 conformation was maintained. G-rich oligomers capable of folding into G4 structures comprise a large group of aptamers with several advantages over other unstructured sequences. G4 structures have demonstrated greater resistance against certain nucleases and are more chemically and thermodynamically stable. Because of their larger negative charge density, they also have better electrostatic interactions with positively charged ligands. Based on the characterization results, BMAA_165_min was identified as the most promising aptamer candidate due to its superior binding affinity, selectivity, and capacity to form highly stable G4 structures.

### Example 4

### Electrochemical Detection of BMAA

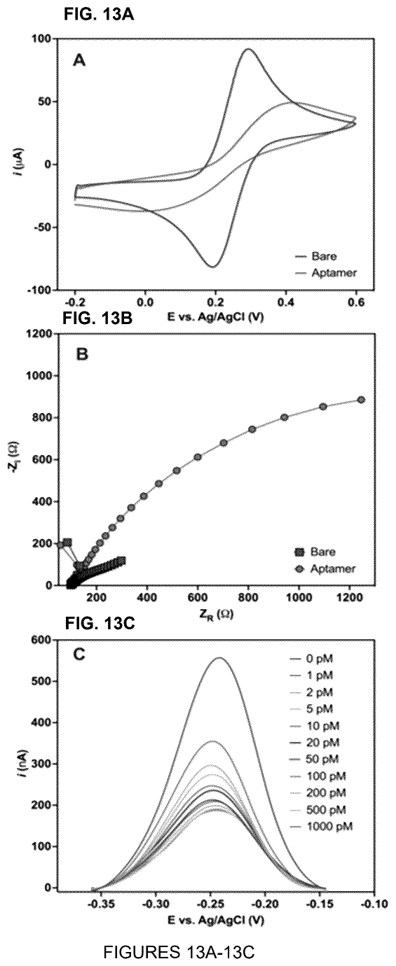

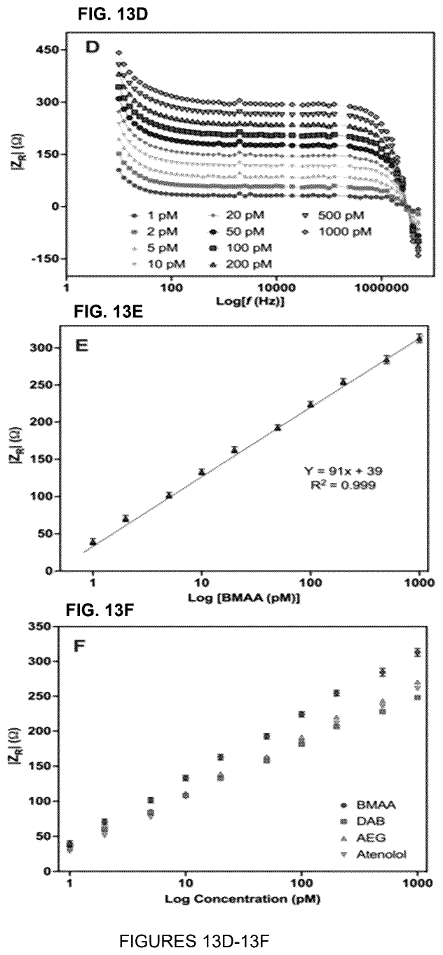

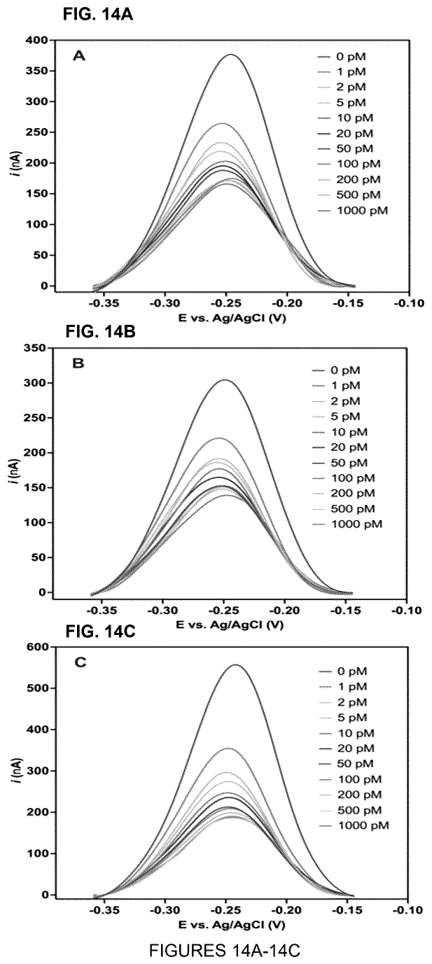

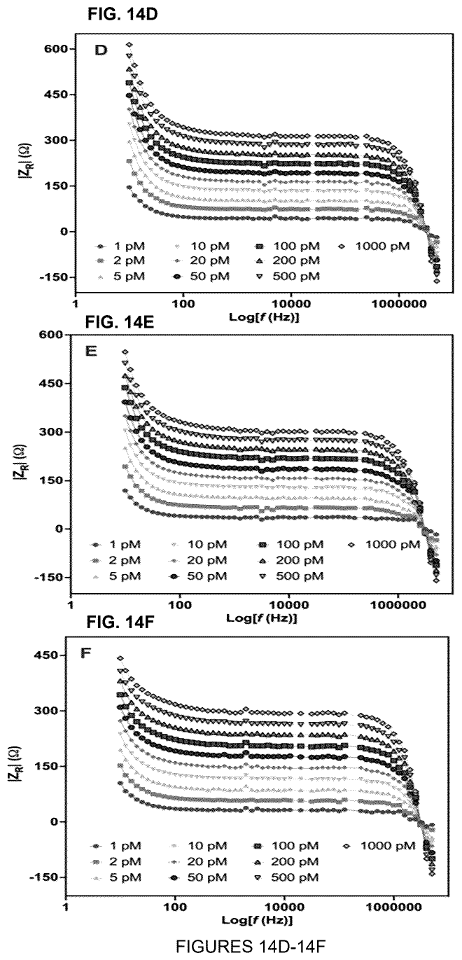

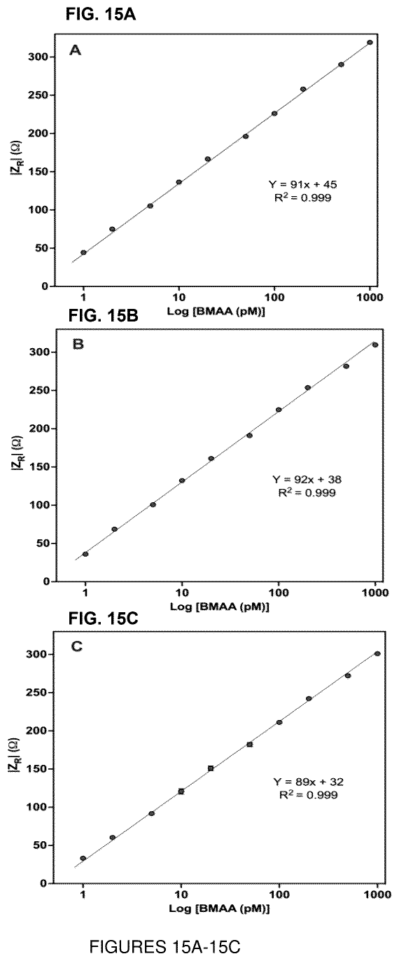

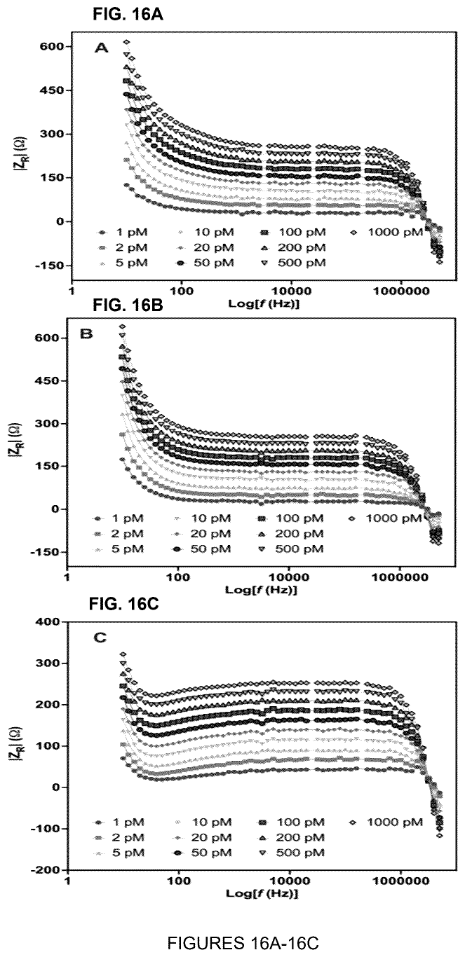

To assess the robustness of the aptamer, a functional validation using methods that reflect the final desired aptamer application is crucial. An electrochemical aptamer-based (EAB) sensor was developed to assess the suitability of BMAA_165_min for biosensing applications. The aptamer molecules were covalently immobilized on the surface of gold (Au) electrodes using thiol-based self-assembled monolayers (SAMs). CV and EIS were used to characterize the aptamer-modified Au electrodes in a 5 mM [Fe(CN)_{6]}^{3-/4-} redox couple solution. The CV of the bare Au electrode shows the characteristic reversible redox peaks, with a peak-to-peak separation (ΔE) of 0.1 V (FIG. 13A). After aptamer immobilization, an increase in ΔE along with a decrease in the cathodic and anodic peak currents was observed. The electrostatic repulsive forces generated by the negative charges of the DNA phosphate backbone slow down the rate of electron transfer between the redox couple molecules and the Au electrode. The Nyquist plots of the aptamer-modified Au electrode shows a significant increase in resistance when compared with the bare Au electrode, which also correlates with slower electron transfer rate (FIG. 13B). The anions in the redox probe encounter more difficulty reaching the Au surface as a result of the partially occupied electrode and the repulsion produced by the negatively charged DNA. The changes observed in both the CV and EIS demonstrate successful attachment of the aptamer on the surface of the Au electrodes.

For the sensing experiments, MB was used as a conjugated redox label for its ability to change due to target-binding induced aptamer folding. MB was covalently linked to aptamers by chemical cross-linking. Additionally, the Au electrodes were blocked with MCH to minimize nonspecific adsorption and avoid the physical adsorption of the DNA aptamers on the electrode surface. The EAB sensor reaction to varying BMAA concentrations was evaluated using SWV and EIS. With SWV, a decrease in the MB current signal with increasing BMAA concentrations was observed (FIG. 13C). This is consistent with a “signal off” system, in which the redox label moves away from the electrode upon target binding, reducing electron flow to the electrode surface. This occurs as a result of the target molecules obstructing the MB redox reaction or the aptamer's conformation changing upon the target. However, whether BMAA_165_min adopts a G4 structure when immobilized in the sensing platform or changes conformation during BMAA binding remains unclear.

Impedance experiments provide more information than voltammetric experiments, making them a more sensitive method of examining alterations to the electrode surface and the electrode-solution interface. EIS was recorded using the redox potential of MB (−0.2 V) at different frequencies. Z_{R} was used as the analytical signal to investigate the EAB sensor's response to varying concentrations of BMAA and determine its analytical range. A concentration-dependent analytical response was observed when the EAB sensor was exposed to BMAA (FIG. 13D). The impedimetric sensor achieved fast (1 minute) quantitative detection of BMAA, yielding a linear response from 1 to 1000 pM (FIG. 13E). The EAB sensor showed high sensitivity and reproducibility with an LOD of 1.13±0.03 PM and an LOQ of 1.46±0.07 pM. It is worth noting that these values are significantly below the expected concentrations of BMAA in water sources. The EAB sensor's selectivity was tested by evaluating its cross-reactivity with the antibiotic atenolol and isomers AEG and DAB. The impedimetric EAB sensor had a higher analytical response for BMAA in comparison to the potentially interfering compounds studied, demonstrating high specificity (FIG. 13F). These findings confirmed the EAB sensor's suitability for the sensitive and selective detection of BMAA, even in the presence of closely related substances like isomers. Moreover, the overall outcomes validate the immobilized aptamer's functionality and its application in biosensing.

TABLE 8
Determination of L.O.D. and L.O.Q. values for the EAB sensor.

| | | | | Mean | | | Mean | |
|---|---|---|---|---|---|---|---|---|
| | | | L.O.D. | L.O.D. | | L.O.Q. | L.O.Q. | |
| Electrode | S | σ | (pM) | (pM) | SEQ | (pM) | (pM) | SE |
| 1 | 91 | 1 | 1.11 | 1.13 | 0.03 | 1.38 | 1.46 | 0.07 |
| 2 | 92 | 2 | 1.17 | | | 1.62 | | |
| 3 | 89 | 1 | 1.11 | | | 1.38 | | |

Simple linear regression analysis was obtained using GraphPad Prism 10.2.0. LOD = 3 s/S and LOQ = 10 s/S, where S is the slope of the curve and s is the standard deviation. SE represents the calculated standard error.

HTS analysis revealed the enrichment of G-rich motifs during the in vitro selection and led to the identification of putative aptamer candidates. From the candidates that displayed BMAA binding, BMAA_159 and BMAA_165 showed the highest affinities. A comprehensive examination of these aptamers through various characterization techniques evidenced the superior specificity and selectivity of BMAA_165 (K_{d}=0.32±0.02 μM) and its truncated form BMAA_165_min (K_{d}=0.63±0.02 μM). Additionally, it was discovered that BMAA_165_min forms highly stable G4 structures. Furthermore, the development of an EAB sensor served to evaluate the suitability of BMAA_165_min in biosensing applications. Fast electrochemical detection of BMAA was achieved with high sensitivity (LOD=1.13±0.02 pM), reproducibility, and selectivity.

1. An aptamer comprising a nucleotide sequence for binding β-N-methylamino-L-alanine (L-BMAA) or an isomer thereof.

2. The aptamer of claim 1, wherein the isomer is selected from the group consisting of (S)-2,4-diaminobutyric acid (L-DAB), N-(2-aminoethyl)glycine (AEG), and β-amino-N-methylalanine (BAMA).

3. The aptamer of claim 1, wherein the nucleotide sequence is a DNA sequence, and wherein the DNA sequence comprises a sequence motif of GAGGGG, GGAGGG, or both.

4. The aptamer of claim 1, wherein the DNA sequence comprises 80% sequence identity to SEQ ID NO: 1 (BMAA_159_min).

5. The aptamer of claim 1, wherein the DNA sequence comprises SEQ ID NO: 1 (BMAA_159_min).

6. The aptamer of claim 1, wherein the DNA sequence comprises 80% sequence identity to SEQ ID NO: 2 (BMAA_165_min).

7. The aptamer of claim 1, wherein the DNA sequence comprises SEQ ID NO: 2 (BMAA_165_min).

8. An electrochemical aptamer-based sensor (EAB) composition comprising a DNA aptamer and a gold nanoparticle, wherein the DNA aptamer is conjugated to the gold nanoparticle.

9. The EAB composition of claim 8, wherein the DNA aptamer is selected from the group consisting of SEQ ID NO: 1 (BMAA_159_min), SEQ ID NO: 2 (BMAA_165_min), SEQ ID NO: 3 (BMAA_159), SEQ ID NO: 4 (BMAA_165), SEQ ID NO: 5 (BMAA_172), SEQ ID NO: 6 (BMAA_96), and SEQ ID NO: 7 (BMAA_38).

10. The EAB composition of claim 8 further comprising a redox label covalently linked to the DNA aptamer.

11. The EAB composition of claim 10, wherein the redox label is methylene blue (MB).

12. A method of detecting β-N-methylamino-L-alanine (L-BMAA) or an isomer thereof in a sample, the method comprising the steps of:
a) contacting the sample with an aptamer comprising a nucleotide sequence for binding L-BMAA to provide a test sample, and
b) performing an assay on the test sample to identify presence of L-BMAA in the sample.

13. The method of claim 12, wherein the isomer is selected from the group consisting of (S)-2,4-diaminobutyric acid (L-DAB), N-(2-aminoethyl)glycine (AEG), and β-amino-N-methylalanine (BAMA).

14. The method of claim 12, wherein the nucleotide sequence is a DNA sequence, and wherein the DNA sequence comprises a sequence motif of GAGGGG, GGAGGG, or both.

15. The method of claim 12, wherein the DNA sequence comprises 80% sequence identity to SEQ ID NO: 1 (BMAA_159_min).

16. The method of claim 12, wherein the DNA sequence comprises SEQ ID NO: 1 (BMAA_159_min).

17. The method of claim 12, wherein the DNA sequence comprises 80% sequence identity to SEQ ID NO: 2 (BMAA_165_min).

18. The method of claim 12, wherein the DNA sequence comprises SEQ ID NO: 2 (BMAA_165_min).

19. The method of claim 12, wherein the sample is selected from the group consisting of an environmental sample, a tissue sample, a food sample, a cyanobacterial sample, a microalgae sample, a plant sample, a zooplankton sample, an animal sample, and a supplement sample.

20. The method of claim 12, wherein the assay comprises square wave voltammetry (SWV), electrochemical impedance spectroscopy (EIS), or both.
